(19) United States
(12) Patent Application Publication
(10) Pub. No.: US 2022/0177831 A1
(43) Pub. Date: Jun. 9, 2022

(54) PRODUCING FUNCTIONAL MICROBIAL CONSORTIA

(71) Applicant: Pluton Biosciences, LLC, St. Louis, MO (US)

(72) Inventors: Steven C. Slater, Vancouver (CA); Barry S. Goldman, Alton, IL (US); Benjamin M. Wolf, St. Louis, MO (US); Ann M. Guggisberg, St. Louis, MO (US); Diana L. Beckman, St. Louis, MO (US); Boahemaa Adu-Oppong, Missouri City, TX (US); Kirk D. Narzinski, Columbia, IL (US)

(21) Appl. No.: 17/544,879

(22) Filed: Dec. 7, 2021

Related U.S. Application Data
(60) Provisional application No. 63/122,889, filed on Dec. 8, 2020.

(51) Int. Cl. C12N 1/20; C12Q 1/689; G06N 3/12

(52) U.S. Cl. CPC C12N 1/20; G06N 3/126; C12Q 1/689

(57) ABSTRACT

Provided herein is technology relating to identifying and isolating microorganisms having a targeted function and particularly, but not exclusively, to methods, compositions, and systems for screening and/or selecting individual microorganisms or microbial consortia that provide specified functions.

This application claims priority to U.S. provisional patent application Ser. No. 63/122,889, filed on Dec. 8, 2020, which is incorporated herein by reference in its entirety.

### FIELD

Provided herein is technology relating to identifying and isolating microorganisms having a targeted function and particularly, but not exclusively, to methods, compositions, and systems for screening and/or selecting individual microorganisms or microbial consortia that provide specified functions.

### BACKGROUND

Various flora and/or fauna may exist and interact in localized, self-sustaining ecosystems called biomes. The operation of a biome comprising communities of flora and/or fauna may impact a local environment or ecosystem. In particular environments, the flora and/or fauna may comprise microorganisms. Although small in physical size, the operation of microorganisms in environments may have substantial effects. For example, yeasts operating in the context of sugar in a closed environment may create alcohol to the point that no more alcohol may be created in the closed system. This can happen either through the exhaustion of sugar or through the quantity of alcohol impeding the creation of more alcohol. At a larger scale, the operation of the flora and/or fauna in a biome may create a global impact beyond a localized ecosystem. For example, Rothschild and Mancinelli, among others, hypothesize that microbial mats and stromatolites contributed to CO₂ fixation and substantively reduced the amount of CO₂ in the Earth's atmosphere during Cambrian times.

There is a desire to search not only for the operation of a single fauna or flora but rather for the operation of fauna and/or flora in concert with each other to optimize for the impact on an environmental variable. However, conventional technologies for identifying and/or isolating microbial organisms are focused on specific phenotypes of individual isolated microorganisms and most conventional technologies are inefficient and slow. Accordingly, there is a need to screen for microbial consortia to optimize variables related to effect on an environment or ecosystem.

### SUMMARY

The term “biomining” refers to searching for organisms meeting predetermined criteria, e.g., using methods comprising screening and/or selecting of organisms. In this context, the term “biomining” as used herein is not to be confused with use of the term in other fields where it describes the use of organisms to extract metals. Conventional methods of biomining are processes that start with a set of known organisms, e.g., microbes, that have known desirable properties. A new set of microbes is identified where the new microbes have similarities to the known organisms, e.g., microbes having phenotypes similar to the initial set of known microbes. The new set is then tested for a specific application. For example, in agriculture, a target species may be a legume being used for a cover crop, and the application is the fixation of nitrogen in the legume stem.

In contrast, the technology provided herein relates to “application-specific biomining” in which the biomining process is inverted with respect to conventional biomining described above. In particular, instead of starting with a set of known microbes as in conventional biomining, application-specific biomining as described herein identifies a target (e.g., species, environment, ecosystem, etc.) that is to be subject to an application of microbial organisms, e.g., for functionally modifying (e.g., improving) the target. The target is then tested against a set of microbial populations that comprise numerous available microbes, which may be known or unknown. In some instances, the set of microbial populations may be subject to a minimal a pre-filtering or a pre-selection prior to the test. As used herein, the term “biomining” refers to “application-specific biomining” as described above and herein unless the context clearly indicates that the term “biomining” refers to conventional biomining.

For example, in various embodiments, a set of microbial populations from the entire set of microbial populations are cultured and applied to a target species for testing. The set of microbial populations applied for testing may include a portion of the entire microbial populations. In instances in which one or more tests for the set of microbial populations show a trend to a desired result with respect to one or more variables under test, the set of microbial populations is selected and sub-cultured, thereby focusing on growing the microbe population most likely causing the desired results. This process may be iterated until the desired causal organisms are identified and isolated.

There are many benefits to application-centric biomining. First, conventional biomining starts with known microbes and new microbes are added for analysis based on perceived “similarity” rather than methodical testing operations. In many cases, an individual microbe may not cause a substantial desired effect on the target, but rather a set comprising two or more microbes acting in concert, known as a “microbial consortium”, causes the desired effect. Accordingly, by starting with a pre-selected set of microbes, an investigator using conventional biomining may inadvertently omit microbes that would provide desired effects in concert with other microbes.

In contrast, embodiments of application-centric biomining provided herein focus on the application and/or functional result to be achieved, e.g., the desired effect as measured by observing variable(s) under test. Thus, the potentially flawed assumption that microbes with similar phenotypes will cause similar desired results is reduced or eliminated. Another benefit is that, in application-specific biomining, the screening process may be much faster and more efficient. For example, in one comparative trial, the amount of time needed to discover desired microbial consortia using application-specific biomining was reduced by half using one-eighth of staff (e.g., a reduction to one-sixteenth of the person-hours), with a corresponding reduction in cost, relative to conventional biomining.

Accordingly, provided herein are embodiments of a method comprising obtaining multiple environmental samples that include organic matter for microbial biomining; mixing the multiple environmental samples into combinations of mixed environmental samples; selecting a particular mixed environmental sample of the mixed environmental samples based on one or more selection criteria for testing; culturing the particular mixed environmental sample as selected in an environment that includes one or more environmental conditions; and in response to determining based on one or more variable measurements that resulted from the culturing that the particular mixed environmental sample produced a successful microbial biomining result, obtaining identification information for microbes that are present in a corresponding microbial consortium of the particular mixed environmental sample. In some embodiments, methods further comprise in response to determining based on the one or more variable measurements that resulted from the culturing that the particular mixed environmental sample produced an unsuccessful microbial biomining result, selecting an additional mixed environmental sample based on the one or more selection criteria for testing. In some embodiments, methods further comprise selecting an additional mixed environmental sample of the mixed environmental samples based on the one or more selection criteria for testing; culturing the additional mixed environmental sample as selected in an environment that includes one or more environmental conditions; and in response to determining based on one or more variable measurements that resulted from the culturing that the additional mixed environmental sample produced an additional successful microbial biomining result, obtaining additional identification information for additional microbes that are present in an additional corresponding microbial consortium of the additional mixed environmental sample. In some embodiments, methods further comprise culturing the corresponding microbial consortium of the particular mixed environmental sample into a microbial culture; growing a selected culture portion of the microbial culture in the environment that includes one or more environmental conditions; and in response to determining based on one or more variable measurements of the selected culture portion that the selected culture portion produced a successful microbial biomining result, obtaining additional identification information for additional microbes that are present in an additional corresponding microbial consortium of the selected culture portion. In some embodiments, methods further comprise in response to determining based on one or more variable measurements of the selected culture portion that the culture portion produced an unsuccessful microbial biomining result, selecting an additional culture portion of the microbial culture for testing. In some embodiments, methods further comprise growing an additional selected culture portion of the microbial culture in the environment that includes one or more environmental conditions; and in response to determining based on one or more variable measurements of the additional selected culture portion that the selected culture portion produced a successful microbial biomining result, obtaining further identification information for further microbes that are present in a further corresponding microbial consortium of the additional selected culture portion.

In some embodiments, methods further comprise generating a machine learning model based on training data that includes the identification information and the additional identification information. In some embodiments, the machine learning model at least correlates one or more environmental sample variable values of the multiple environmental samples with microbial variable values of one or more microbial species and one or more microbial consortia that are present in the multiple environmental samples. In some embodiments, the methods further comprise receiving a request for information related to one or more variable values, and applying the machine learning model to the one or more variable values to at least one of identifying one or more microbial species that are associated with the one or more variable values, identifying one or more environmental characteristics that are associated with the one or more variable values, or identifying at least one microbial consortium that is associated with the one or more variable values. In some embodiments, the one or more variable values may include at least one of a phenotype of a microbe, a desired amount of nitrogen fixation, a desired amount of carbon sequestration, one or more environmental sample characteristics, or one or more variables (e.g., one or more climate change variables). In some embodiments, the one or more environmental characteristics include an environmental source location and an environmental composition. In some embodiments, the machine learning model further correlates one or more environmental sample variable values and microbial variable values with one or more variables (e.g., climate change variable values), and wherein the one or more variable values (e.g., climate change variable values) include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass. In some embodiments, the one or more environmental conditions include at least one of a particular concentration of N₂ gas, a particular concentration of CO₂ gas, availability of one or more specific nutrients, availability of one or more specific salts, or availability of one or more specific additives. In some embodiments, the one or more variable measurements includes a variable measurement that indicates an increase in carbon sequestration, an increase in nitrogen fixation, an increase in biomass, or having a microbe that is able to meet a particular survival time. In some embodiments, a successful microbial biomining result is produced when each variable measurement of one or more variable measurements at least met a corresponding variable measurement threshold. In some embodiments, the identification information of a microbe includes a DNA biomarker of the microbe.

In some embodiments, the technology provides one or more non-transitory computer-readable media storing computer-executable instructions that upon execution cause one or more processors to perform acts comprising generating a machine learning model that based on training data that includes the identification information of one or more microbes, the machine learning model at least correlating one or more environmental sample variable values of multiple environmental samples with microbial variable values of one or more microbial species and one or more microbial consortia that are present in the multiple environmental samples; receiving a request for information related to one or more variable values; and applying the machine learning model to the one or more variable values to at least one of identifying one or more microbial species that are associated with the one or more variable values, identifying one or more environmental characteristics that are associated with the one or more variable values, or identifying at least one microbial consortium that is associated with the one or more variable values. In some embodiments, the one or more variable values may include at least one of a phenotype of a microbe, a desired amount of nitrogen fixation, a desired amount of carbon sequestration, one or more environmental sample characteristics, or one or more variables (e.g., climate change variables). In some embodiments, the machine learning model further correlates one or more environmental sample variable values and microbial variable values with one or more variable values (e.g., climate change variable values), and wherein the one or more variable values (e.g., climate change variable values) include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

In some embodiments, the technology provides a computing device comprising one or more processors; and memory including a plurality of computer-executable components that are executable by the one or more processors to perform a plurality of actions, the plurality of actions comprising generating a machine learning model that, based on training data that includes the identification information of one or more microbes, the machine learning model at least correlates one or more environmental sample variable values of multiple environmental samples with microbial variable values of one or more microbial species and one or more microbial consortia that are present in the multiple environmental samples; receiving a request for information related to one or more variable values; and applying the machine learning model to the one or more variable values to at least one of identifying one or more microbial species that are associated with the one or more variable values, identifying one or more environmental characteristics that are associated with the one or more variable values, or identifying at least one microbial consortium that is associated with the one or more variable values.

In some embodiments, the technology provides methods comprising obtaining an environmental sample comprising organic matter for microbial biomining; homogenizing the environmental sample to produce an input sample; culturing the input sample in an environment that includes one or more environmental conditions; and in response to determining based on one or more variable measurements that resulted from the culturing that the input sample produced a successful microbial biomining result, obtaining identification information for microbes that are present in a corresponding microbial consortium of the input sample. In some embodiments, methods comprise obtaining a plurality of environmental samples comprising organic matter for microbial biomining; homogenizing each environmental sample to produce a plurality of input samples; and selecting an input sample from the plurality of input samples. In some embodiments, methods further comprise in response to determining based on the one or more variable measurements that resulted from the culturing that the input sample produced an unsuccessful microbial biomining result, producing a second input sample based on the one or more selection criteria for testing.

In some embodiments, methods further comprise producing a second input sample based on the one or more selection criteria for testing; culturing the second input sample as selected in an environment that includes one or more environmental conditions; and in response to determining based on one or more variable measurements that resulted from the culturing that the second input sample produced an additional successful microbial biomining result, obtaining additional identification information for additional microbes that are present in a second corresponding microbial consortium of the second input sample. In some embodiments, methods further comprise culturing the corresponding microbial consortium of the input sample into a microbial culture; growing a selected culture portion of the microbial culture in the environment that includes one or more environmental conditions; and in response to determining based on one or more variable measurements of the selected culture portion that the selected culture portion produced a successful microbial biomining result, obtaining additional identification information for additional microbes that are present in an additional corresponding microbial consortium of the selected culture portion. In some embodiments, methods further comprise in response to determining based on one or more variable measurements of the selected culture portion that the culture portion produced an unsuccessful microbial biomining result, selecting an additional culture portion of the microbial culture for testing. In some embodiments, methods further comprise growing an additional selected culture portion of the microbial culture in the environment that includes one or more environmental conditions; and in response to determining based on one or more variable measurements of the additional selected culture portion that the selected culture portion produced a successful microbial biomining result, obtaining further identification information for further microbes that are present in a further corresponding microbial consortium of the additional selected culture portion.

In some embodiments, methods further comprise generating a machine learning model based on training data that includes the identification information and the additional identification information. In some embodiments, the machine learning model at least correlates one or more environmental sample variable values of the environmental sample with microbial variable values of one or more microbial species and one or more microbial consortia that are present in the environmental sample. In some embodiments, methods further comprise receiving a request for information related to one or more variable values, and applying the machine learning model to the one or more variable values to at least one of: identifying one or more microbial species that are associated with the one or more variable values; identifying one or more environmental characteristics that are associated with the one or more variable values; and/or identifying at least one microbial consortium that is associated with the one or more variable values. In some embodiments, the one or more variable values may include at least one of a phenotype of a microbe, a desired amount of nitrogen fixation, a desired amount of carbon sequestration, one or more environmental sample characteristics, and/or one or more variables. In some embodiments, the one or more environmental characteristics include an environmental source location and an environmental composition. In some embodiments, the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass. In some embodiments, the one or more environmental conditions include at least one of a particular concentration of N₂ gas, a particular concentration of CO₂ gas, availability of one or more specific nutrients, availability of one or more specific salts, or availability of one or more specific additives. In some embodiments, the one or more variable measurements includes a variable measurement that indicates an increase in carbon sequestration, an increase in nitrogen fixation, an increase in biomass, or having a microbe that is able to meet a particular survival time. In some embodiments, a successful microbial biomining result is produced when each variable measurement of one or more variable measurements at least met a corresponding variable measurement threshold. In some embodiments, the identification information of a microbe includes a DNA biomarker of the microbe.

In some embodiments, the technology provides one or more non-transitory computer-readable media storing computer-executable instructions that upon execution cause one or more processors to perform acts comprising generating a machine learning model that based on training data that includes the identification information of one or more microbes, the machine learning model at least correlating one or more environmental sample variable values of an environmental sample with microbial variable values of one or more microbial species and one or more microbial consortia that are present in the environmental sample; receiving a request for information related to one or more variable values; and applying the machine learning model to the one or more variable values to at least one of; identifying one or more microbial species that are associated with the one or more variable values; identifying one or more environmental characteristics that are associated with the one or more variable values; and/or identifying at least one microbial consortium that is associated with the one or more variable values. In some embodiments, the one or more variable values may include at least one of a phenotype of a microbe, a desired amount of nitrogen fixation, a desired amount of carbon sequestration, one or more environmental sample characteristics, or one or more variables. In some embodiments, the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

In some embodiments, the technology provides a computing device, comprising one or more processors; and a memory including a plurality of computer-executable components that are executable by the one or more processors to perform a plurality of actions, the plurality of actions comprising generating a machine learning model that, based on training data that includes the identification information of one or more microbes, the machine learning model at least correlates one or more environmental sample variable values of an environmental sample with microbial variable values of one or more microbial species and one or more microbial consortia that are present in the environmental sample; receiving a request for information related to one or more variable values; and applying the machine learning model to the one or more variable values to at least one of: identifying one or more microbial species that are associated with the one or more variable values; identifying one or more environmental characteristics that are associated with the one or more variable values; and/or identifying at least one microbial consortium that is associated with the one or more variable values.

Furthermore, in some embodiments, the technology provides a method for producing a microbial consortium that performs a specified function. For example, in some embodiments, methods comprise providing a sample comprising a plurality of microorganisms; inoculating a first volume of a growth medium with a portion of said sample to provide a first culture; growing the first culture under a set of selective conditions; producing a first taxonomic classification of microorganisms in the first culture; inoculating a second volume of the growth medium with a portion of the first culture to provide a second culture; growing the second culture under the set of selective conditions; producing a second taxonomic classification of microorganisms in the second culture; and deriving a measure of microbial community stability of the second culture with respect to the first culture using the second taxonomic classification and the first taxonomic classification.

In some embodiments, the technology provides an iterative and/or recursive method where steps are repeated until a monitored measured characteristic reaches a specified value and/or reaches a plateau. For instance, in some embodiments, the technology provides a method for producing a microbial consortium that performs a specified function, the method comprising providing a sample comprising a plurality of microorganisms; inoculating an Nth volume of a growth medium with a portion of said sample to provide an Nth culture; growing the Nth culture under a set of selective conditions; producing an Nth taxonomic classification of microorganisms in the Nth culture; inoculating a N+1th volume of the growth medium with a portion of the Nth culture; growing the N+1th culture under the set of selective conditions; producing a N+1th taxonomic classification of microorganisms in the N+1th culture; deriving a measure of microbial community stability of the N+1th culture with respect to the Nth culture using the N+1th taxonomic classification and the Nth taxonomic classification; repeating iteratively, with the N+1th culture acting as the Nth culture, the steps of inoculating a N+1th volume of the growth medium with a portion of the Nth culture; growing the N+1th culture under the set of selective conditions; producing a N+1th taxonomic classification of microorganisms in the N+1th culture; and deriving a measure of microbial community stability of the N+1th culture with respect to the Nth culture using the N+1th taxonomic classification and the Nth taxonomic classification until the measure of microbial community stability reaches a plateau value; and providing the stable N+1th culture as comprising a microbial consortium that performs a specified function. In some embodiments, the sample is an environmental sample. In some embodiments, the environmental sample is a soil or water sample. In some embodiments, the growth medium and/or selective conditions select for the specified function. In some embodiments, producing a taxonomic classification comprises obtaining metagenomic nucleotide sequence data for a culture and identifying taxonomic units present in the culture using analysis of the metagenomic nucleotide sequence data. In some embodiments, the microbial consortium comprises a number of taxonomic units that is at least 2, 3, 4, 5, or 6. In some embodiments, a microbial community having a number of taxonomic units that is less than the number of taxonomic units of the microbial consortium does not perform the specified function. In some embodiments, any one of the taxonomic units alone does not perform the specified function. In some embodiments, the measure of microbial community stability comprises a measure of richness, diversity, abundance, and/or membership. In some embodiments, the growing occurs for an empirically determined time for growth to end of exponential phase. In some embodiments, the method further comprises measuring the growth rate of the Nth or N+1th culture. In some embodiments, a growth rate is determined by measuring cell mass as a function of time. In some embodiments, at least one of the taxonomic units does not grow as a pure culture in the culture medium under the selective conditions. In some embodiments, a microbial community comprising a number of taxonomic units that is at least two and that is less than the number of taxonomic units of the microbial consortium does not grow in the culture medium under the selective conditions.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings.

FIG. 1 illustrates an example environment for the application-centric microorganism screening.

FIG. 2 is a block diagram showing various components of one or more illustrative computing devices that supports the use of machine learning techniques with respect to application-centric microorganism screening.

FIGS. 3a and 3b illustrate a flow diagram of an example process for performing the application-centric microorganism screening.

FIG. 4 is a flow diagram of an example process for using machine learning techniques to identify microbial species and other information related to one or more variables.

FIG. 5 is a flow diagram of an example process for producing a microbial consortium that performs a specified function.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to identifying and isolating microorganisms having a targeted function and particularly, but not exclusively, to methods, compositions, and systems for screening and/or selecting individual microorganisms or microbial consortia that provide specified functions.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

Unless otherwise defined herein, scientific and technical terms used in connection with the present technology shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present technology are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Sambrook et al., Molecular Cloning: A Laboratory Manual, 3d ed., Cold Spring Harbor Laboratory Press, Cold Spring Harbor, N.Y. (2000); Ausubel et al., Current Protocols in Molecular Biology, Greene Publishing Associates (1992 and Supplements to 2000); Ausubel et al., Short Protocols in Molecular Biology: A Compendium of Methods from Current Protocols in Molecular Biology, 4th ed., Wiley & Sons (1999); Harlow and Lane, Antibodies: A Laboratory Manual, Cold Spring Harbor Laboratory Press, Cold Spring Harbor, N.Y. (1990); Harlow and Lane, Using Antibodies: A Laboratory Manual, Cold Spring Harbor Laboratory Press, Cold Spring Harbor, N.Y. (1998); and T. Kieser et al., Practical Streptomyces Genetics, John Innes Foundation, Norwich (2000); each of which is incorporated herein by reference in its entirety.

As used herein, the term “culturable organism” refers to a living organism that can be maintained and grown in a laboratory. In some embodiments, a culturable organism may not be maintained and grown in a laboratory in a pure culture free of other organisms and so may be referred to as an “unculturable organism” with respect to growing as a pure culture. However, in some embodiments, such an organism may be grown in a laboratory in a microbial consortium comprising at least one other organism and so may be a “culturable organism” with respect to the consortium and be also an “unculturable organism” with respect to being grown in a pure culture without the other member(s) of the consortium.

As used herein, the terms “selected environment”, “condition”, or “conditions” refer to any external property in which a particular organism or a microbial consortium of a microbial community grows more efficiently (e.g., faster, to a higher amount or concentration, with greater survival, etc.) than one or more other organisms or consortia of the microbial community. Exemplary “conditions” or “environments” include, but are not limited to, a particular medium, volume, vessel, temperature, mixing, aeration, gravity, electromagnetic field, cell density, pH, nutrients, phosphate source, nitrogen source, symbiosis with one or more organisms, and/or interaction with a single species of organism or multiple species of organisms (e.g., a mixed population). Also included as “conditions” or “environments” are substances that may be toxic to one or more organisms or consortia of a microbial community, such as heavy metals, antibiotics, and chlorinated compounds. It should be understood that time may also be considered a “condition” since organisms are not static entities. Thus, a culture grown over an extended period of time (e.g., days, weeks, months, years) may produce a culture comprising a particular organism or a consortium at a relatively higher proportion in the culture than the relative amount of the particular organism or the consortium in the culture prior to the growth for the time period.

As used herein, the term “selection” refers to an increase in the frequencies of different “types” of individuals within a population by removal or enrichment of some types more so than others, either intentionally or spontaneously. The nature of a “type” can be defined by genetic characterization (e.g., genes or nucleotide sequences); functional characterization (e.g., enzymatic, metabolic ability); taxonomic characterization (e.g., strain, subspecies, species, genus, family, or an operational taxonomic unit (OTU) based on nucleotide sequence similarity or difference); or by physical characterization. Furthermore, a type may comprise one or many individuals. An archetypal example of selection includes, but is not limited to growth rate selection, in which individuals that grow and reproduce more quickly become more prevalent in a population. An important consideration in conducting selection is to determine what the “selection is for” or what is “being selected,” that is to say, the genetic, functional, and/or physical difference that is favorable or unfavorable in a particular environment. Growth rate selection is applied to select organisms having a growth rate that is faster than other individuals in the population and that can be passed from a parent cell to its offspring.

As used herein, the term “enrichment” refers to a process wherein the abundance (e.g., expressed in absolute and/or relative terms) of one or more organism(s), one or more functional ability(ies), one or more gene(s) or gene product(s), or one or more nucleotide sequence(s) of interest is/are increased relative to the abundance of one or more other organism(s), one or more other functional ability(ies), one or more other gene(s) or gene product(s), or one or more other nucleotide sequence(s). For example, in some embodiments, the term “enrichment” refers to a process of increasing the number (e.g., the absolute and/or relative number) of one or more microorganisms present in a culture, e.g., by culturing in a suitable medium under selective conditions.

As used herein, the term “medium” or “media” refers to the chemical environment to which an organism is subjected or is provided access. The organism may either be immersed within the media or be within physical proximity (e.g., physical contact) thereto. Media typically comprise water with other additional nutrients and/or chemicals that may contribute to the growth or maintenance of an organism. The ingredients may be purified chemicals (e.g., a “defined” media) or complex, uncharacterized mixtures of chemicals such as extracts made from milk or blood. Standardized media are widely used in laboratories. Examples of media for the growth of bacteria include, but are not limited to, LB and M9 minimal medium. The term “minimal” when used in reference to media refers to media that support the growth of an organism but are composed of only the simplest possible chemical compounds. For example, an M9 minimal medium may be composed of the following ingredients dissolved in water and sterilized: 48 mM Na₂HPO₄, 22 mM KH₂PO₄, 9 mM NaCl, 19 mM NH₄Cl, 2 mM MgSO₄, 0.1 mM CaCl₂, 0.2% carbon and energy source (e.g., glucose).

As used herein, the term “culture” refers to medium in a container or enclosure with at least one cell or individual of a viable organism, usually a medium in which that organism can grow. As used herein, the term “continuous culture” is intended to mean a liquid culture into which new medium is added at some rate equal to the rate at which medium is removed. Conversely, a “batch culture,” as used herein, is intended to mean a culture of a fixed size or volume to which new media is not added or removed.

As used herein, the term “genetic basis” refers to the underlying genetic or genomic cause of a particular observation.

As used herein, the term “genetic” refers to the heritable information encoded in the sequence of DNA nucleotides. As such, the term “genetic characterization” is intended to mean the sequencing, genotyping, comparison, mapping, or other assay of information encoded in DNA.

As used herein, the term “genetic material” refers to the DNA within an organism that is passed along from one generation to the next. Normally, genetic material refers to the genome of an organism. Extra-chromosomal elements, such as organelle or plasmid DNA, can also be a part of the genetic material that determines organism properties.

As used herein, the term “genetic change” or “genetic adaptation” refers to one or more mutations within the genome of an organism. As used herein, the term “mutation” refers to a difference in the sequence of DNA nucleotides of two related organisms, including substitutions, deletions, insertions and rearrangements, or motion of mobile genetic elements, for example.

As used herein, the term “evaluation” is intended to mean observations or measurements of an observable phenotype of an organism. Evaluation typically includes analysis, interpretation, and/or comparison with the phenotype of another organism. It should be understood that a phenotype may be evaluated at both the genetic level (e.g., with respect to nucleotide sequence) and at the level of gene products. Further, a phenotype may be evaluated in terms of the behavior of the organism within the environment and/or the behavior of individual molecules or groups of molecules within the organism. Such comparisons are useful in determining the detailed function of mutated products resulting from genetic adaptation.

As used herein, the term “step-wise” is intended to mean in the fashion of a series of events, one following the other in time. As used herein, the term “simultaneous” is intended to mean happening at the same time.

As used herein, the terms “microbial”, “microbial organism”, and “microorganism” refer to an organism that exists as a microscopic cell that is included within the domains of Archaea, Bacteria, or Eukarya in the three-domain system (see Woese (1990) Proc Natl Acad Sci USA 87: 4576-79, incorporated herein by reference), the latter including yeast and filamentous fungi, protozoa, algae, or higher Protista. Therefore, the term is intended to encompass prokaryotic or eukaryotic cells or organisms having a microscopic size and includes bacteria, archaea, and eubacteria of all species as well as eukaryotic microorganisms such as yeast and fungi. Also included are cell cultures of any species that can be cultured for the production of a chemical.

As described herein, in some embodiments, microorganisms are prokaryotic microorganisms. In some embodiments, the prokaryotic microorganisms are bacteria. “Bacteria”, or “eubacteria”, refers to a domain of prokaryotic organisms. Bacteria include at least eleven distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles. “Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium. “Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “naturally occurring”, when used in reference to a microorganism, is intended to mean a microorganism that is found in nature. For example, a naturally occurring organism can be isolated from a source in nature and has not been intentionally modified by a human in the laboratory.

As used herein, the term “non-naturally occurring” as applied to a microorganism refers to a microorganism comprising at least one genetic alteration not normally found in the naturally occurring microorganism. Genetic alterations include, for example, modifications introducing expressible nucleic acids encoding metabolic polypeptides, other nucleic acid additions, nucleic acid deletions, and/or other functional disruption of the microbial genetic material. Such modifications include, for example, coding regions and functional fragments thereof, for heterologous, homologous, or both heterologous and homologous polypeptides for the referenced species. Additional modifications include, for example, non-coding regulatory regions in which the modifications alter expression of a gene or operon.

As used herein, the term “microbial consortium” (plural “microbial consortia”) refers to a set of microbial species, or strains of a species, that can be described as carrying out a common function, or can be described as participating in, or leading to, or correlating with, a recognizable parameter or phenotypic trait. A consortium may comprise two or more taxonomic units (e.g., families, genera, species, or strains of a species) of microbes. In some instances, the microbes coexist within the community symbiotically. A microbial consortium may be described by describing taxonomic units present in the consortium (e.g., a number of strains, subspecies, species, genera, families, or operational taxonomic units (OTUs) based on nucleotide sequence similarity or difference); by describing genes present in the consortium; by describing nucleotide sequences present in the consortium; or by describing functions present in and/or provided by the consortium. A microbial consortium may be a subset of organisms found in a microbial community.

As used herein, the term “microbial community” refers to a group of microbes comprising two or more taxonomic units (e g, families, genera, species, or strains of a species) of microbes. Unlike a microbial consortium, a microbial community does not necessarily act in concert to carry out a common function, or does not have to be participating in, or leading to, or correlating with, a recognizable parameter or phenotypic trait.

As used herein, the term “metagenome” is defined as “the collective genomes of all microorganisms present in a given habitat” (Handelsman et al., (1998) Chem. Biol. 5: R245-R249). This term is also intended to include nucleic acids extracted from a microbial community or a microbial consortium (e.g., from an environmental sample) as being representative of the microbial community or microbial consortium, regardless of whether all genomic nucleic acids of the microbial community or microbial consortium are extracted or not.

As used herein, the term “taxonomic unit” is a group of organisms that are considered similar enough to be treated as a separate unit. A taxonomic unit may comprise a family, genus, species, or population within a species (e.g., strain), but is not limited as such.

As used herein, the term “operational taxonomic unit” (OTU) refers to a group of microorganisms considered similar enough to be treated as a separate unit. An OTU may comprise a taxonomic family, genus, or species but is not limited as such. OTUs are frequently defined by comparing nucleotide sequences between organisms. In certain cases, the OTU may include a group of microorganisms treated as a unit based on, e.g., a sequence identity of ≥97%, ≥95%, ≥90%, ≥80%, or ≥70% among at least a portion of a differentiating biomarker, such as the 16S rRNA gene.

The term “genus” may be defined as a taxonomic group of related species according to the Taxonomic Outline of Bacteria and Archaea (Garrity et al. (2007) The Taxonomic Outline of Bacteria and Archaea. TOBA Release 7.7, March 2007. Michigan State University Board of Trustees). The term “species” may be defined as a collection of closely related organisms with greater than 97% 16S ribosomal RNA sequence homology and greater than 70% genomic hybridization and sufficiently different from all other organisms so as to be recognized as a distinct taxonomic unit.

As used herein, the term “relative abundance” refers to the abundance of microorganisms of a particular taxonomic unit (e.g., an OTU) in a first biological sample compared to the abundance of microorganisms of the corresponding taxonomic unit in one or other (e.g., second) samples. The “relative abundance” may be reflected in, e.g., the number of isolated species corresponding to a taxonomic unit or the degree to which a biomarker (e.g., a nucleotide sequence) specific for the taxonomic unit is present or expressed in a given sample. The relative abundance of a particular taxonomic unit in a sample can be determined using culture-based methods or non-culture-based methods well known in the art. Non-culture based methods include sequence analysis of amplified polynucleotides specific for a taxonomic unit or a comparison of proteomics-based profiles in a sample reflecting the number and degree of polypeptide-based, lipid-based, polyssacharide-based or carbohydrate-based biomarkers characteristic of one or more taxonomic units present in the samples. Relative abundance or abundance of taxonomic units or OTU can be calculated with reference to all taxonomic units/OTU detected, or with reference to some set of invariant taxonomic units/OTUs. In some embodiments, taxonomic units are identified using sequence based methods as described in, e.g., Wood (2014) “Kraken: ultrafast metagenomic sequence classification using exact alignments” Genome Biology 15: R46 and Wood (2019) “Improved metagenomic analysis with Kraken 2” Genome Biology 20:257, each of which is incorporated herein by reference.

As used herein, the term “significantly altered relative abundance” refers to a statistically significant increase or reduction in the relative abundance of the number of microorganisms of a particular taxonomic unit compared to the total microorganisms in the sample or to the number of microorganisms of the corresponding taxonomic unit present in another sample. In some embodiments, a “significant increase” or “significant reduction” in relative abundance is defined as a statistically significant increase or statistically significant reduction over a reference value. In some embodiments, a statistically significant increase or statistically significant reduction is an increase or a reduction that is twice, three-times, or four-times of the standard deviation of the relative abundance. In some embodiments, a statistically significant increase or statistically significant reduction is an increase or a reduction with a P-value equal to, or smaller than, 0.1, 0.05, 0.01, or 0.005.

In some embodiments, “significant reduction” or “significant increase” in relative abundance means a statistically significant difference in one or more indicator species or taxonomic unit compared with each other or with reference species or taxonomic units using a non-parametric statistical test, such as a signed-rank test. In some embodiments, a “significant reduction” or “significant increase” in relative abundance is determined using models that employ Bayesian inference and related approaches.

In certain embodiment, an increase in relative abundance reflects an increase of 10%, 20%, 30%, 40%, 50%, 60%, 70%, 80%, 90%, 100% or more over a reference value. In some embodiments, an increase in relative abundance reflects a 2-fold, 3-fold, 5-fold, 10-fold, 20-fold, 50-fold, or 100-fold increases over a reference value.

As used herein, “isolate”, “isolated”, “isolated microbe”, and like terms are intended to mean that the one or more microorganisms has been separated from at least one of the materials with which it is associated in a particular environment (for example, soil, water, or a higher multicellular organism). Thus, an “isolated microbe” does not exist in its naturally occurring environment; rather, through the various techniques described herein, the microbe has been removed from its natural setting and placed into a non-naturally occurring state of existence. Thus, the isolated strain may exist as, for example, a biologically pure culture, or as spores (or other forms of the strain) in association with a carrier composition. In certain aspects of the disclosure, the isolated microbes exist as isolated and biologically pure cultures. It will be appreciated by one of skill in the art that an isolated and biologically pure culture of a particular microbe denotes that said culture is substantially free (within scientific reason) of other living organisms and contains only the individual microbe in question. The culture can contain varying concentrations of said microbe, and isolated and biologically pure microbes often necessarily differ from less pure or impure materials. Furthermore, in some aspects, the disclosure provides for certain quantitative measures of the concentration, or purity limitations, that are found within an isolated and biologically pure microbial culture. The presence of these purity values, in certain embodiments, is a further attribute that distinguishes the presently disclosed microbes from those microbes existing in a natural state.

As used herein, the term “improved” refers to improving a characteristic of an environment as compared to a control environment or as compared to a known average quantity associated with the characteristic in question. For example, “improved” soil may refer to a soil that increases the production of plant biomass after application of a beneficial microorganism or microbial consortium to the soil relative to the plant biomass produced by soil not treated with the beneficial microorganism or microbial consortium and for which other soil characteristics are substantially and/or essentially the same with respect to effects on production of plant biomass. Alternatively, one could compare the production of plant biomass after application of a beneficial microorganism or microbial consortium to the soil relative to the average biomass normally produced by the soil, as represented in scientific or agricultural publications known to those of skill in the art. As used herein, “improved” does not necessarily demand that the data be statistically significant (e.g., p<0.05); rather, any quantifiable difference demonstrating that one value (e.g. the average treatment value) is different from another (e.g. the average control value) can rise to the level of “improved.”

As used herein, the term “phenotype” refers to the observable characteristics of an individual cell, cell culture, organism, or group of organisms (e.g., microbial consortium) that results from the interaction between the genetic makeup (e.g., genotype) of the individual cell, cell culture, organism, or group of organisms and the environment.

In some embodiments, a microbe can be “endogenous” to an environment. As used herein, a microbe is considered “endogenous” to an environment if the microbe is derived from the environment from which it is sourced. That is, if the microbe is naturally found associated with said environment then the microbe is endogenous to the environment. In embodiments in which an endogenous microbe is applied to an environment, then the endogenous microbe is applied in an amount that differs from the levels found in the specified environment in nature. Thus, a microbe that is endogenous to a given environment can still improve the environment if the microbe is present in the environment at a level that does not occur naturally and/or if the microbe is applied to the environment with other organisms that are exogenous to the environment and/or endogenous to the environment and present at a level that does not occur naturally.

In some embodiments, a microbe can be “exogenous” (also termed “heterologous”) to an environment. As used herein, a microbe is considered “exogenous” to an environment if the microbe is not derived from the environment from which it is sourced. That is, if the microbe is not naturally found associated with the environment, then the microbe is exogenous to the environment. For example, a microbe that is normally associated with a first environment may be considered exogenous to a second environment that naturally lacks said microbe.

As used herein, “environmental sample” means a sample taken or acquired from any part of the environment (e.g., ecosystem, ecological niche, habitat, etc.) An environmental sample may include liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present method.

### Description

Provided herein is technology relating to identifying and isolating microorganisms having a targeted function and particularly, but not exclusively, to methods, compositions, and systems for screening and/or selecting individual microorganisms or microbial consortia that provide specified functions. In some ways, the technology presents a problem related to a desired function to a microbial community, where survival or increased prevalence of members of the community depends on one or more members of the community responding with a functional solution. The genetic basis of the solution does not matter, just the relevant property of the one or more members in the responding organism or microbial consortium. Therefore, selection is not biased to a particular set of genes and does not rely on current knowledge.

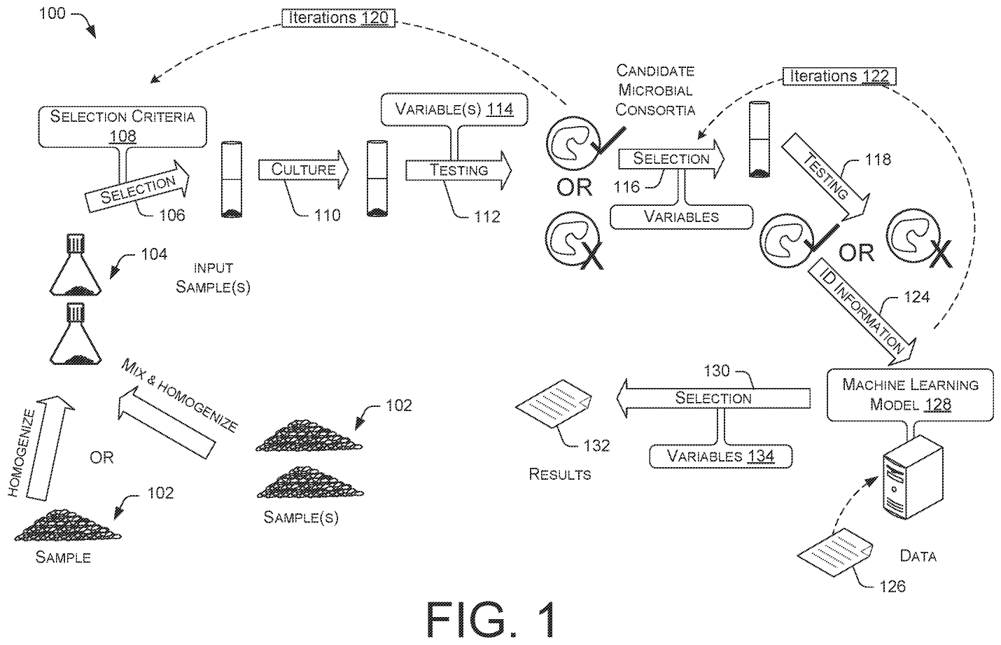

For example, e.g., as shown in FIG. 1, embodiments of the technology relate to methods comprising microorganism screening for biomining. In particular, FIG. 1 illustrates an example environment 100 for the application-centric microorganism screening (e.g., for effective climate change variable and biomining). Because application-centric biomining focuses on an application (e.g., functional) result as measured via a variable under test, rather than on individual microbial phenotypes, the desired application result need not be limited to a local ecosystem. Accordingly, application-centric biomining may be used to identify microbial consortia that may result in changes in more specific and/or more general environments including, but not limited to, microenvironments, species-related microbiomes, ecosystems, local environments, and global environments.

One class of applications relates to impacting climate change variables. It is well known in science that, since the industrial revolution, atmospheric CO₂ levels have been steadily increasing and contributing to global warming. It is well understood to use biomes to lower atmospheric CO₂ levels. As previously stated, there are hypotheses that stromatolites significantly contributed to lowering atmosphere CO₂ during the Cambrian. It is understood that cutting down forest biomes, such as the Amazon, eliminates CO₂ sinks, which in turn contributes to the increase of atmospheric CO₂. Accordingly, this suggests that microbial consortia that can impact climate change may be discovered faster and more efficiently through application-centric biomining.

There are many ways to address global warming, each with a set of variables that can be tested for desired results in application-centric biomining. Such variables that relate to global warming or climate change are referred to herein as climate change variables. One specific application is the use of microbial consortia to maximize sequestration of CO₂ in biomass. Accordingly, the absolute amount of CO₂ and the mass ratio of biomass to sequestered CO₂ are candidate climate change variables, e.g., providing an application or functional result to be sought. As these variables relate to desired end results, these climate change variables may be mathematically treated as statistically independent variables. It is also observed that sequestration of CO₂ in the soil is related to nitrogen fixation. Accordingly, an absolute amount of nitrogen fixation and mass ratio of biomass to fixed nitrogen may be mathematically treated as statistically dependent variables.

Because the desired results are application-specific, the variables being tested, e.g., climate change-related or otherwise, need not be measures of biology or chemical factors. A desired result may be economic. In one example, an application may be maximizing income for performing CO₂ sequestration. Specifically, with increased awareness around global warming, economic markets around carbon credits as well as direct payments for CO₂ sequestration have developed. Accordingly, economic climate change variables may include total profit derived from CO₂ sequestration (e.g., income from CO₂ sequestration less the costs of performing the microbial consortia application).

Some climate change variables may be desirable to measure to provide support for holistic analyses regarding the benefits and disadvantages of CO₂ sequestration. For example, CO₂ sequestration may be accomplished by growing forests of trees. However, trees themselves cannot be eaten for food by humans. Accordingly, a climate change variable may include ratios of the mass of food produced against the mass of CO₂ sequestered. Similarly, CO₂ sequestration in biomass appears to be short-term (e.g., 1-2 years due to outgassing during decomposition). Accordingly, a climate change variable may include the time persistence of CO₂ sequestration.

In various embodiments, application-centric biomining may be performed based on one or more climate change variables. For example, the climate change variables may be measurements for CO₂ sequestration, nitrogen fixation, and survival time/persistence of the microbes. Such an example is based on the understanding that cyanobacteria (photosynthetic microbes) are highly effective at consuming CO₂, and that other microbes, such as Azotobacter vinelandii, are able to fix nitrogen to support carbon sequestration and increase organic matter in biomass.

During the application-centric biomining, one or more environmental samples 102 (e.g., environmental samples that are high in organic matter) may be collected. If a single environmental sample 102 is collected, methods comprise homogenizing the environmental sample to provide an input sample for the application-centric biomining (see, e.g., FIG. 1). If a plurality of environmental samples 102 is collected, methods comprise mixing the plurality of environmental samples to provide a mixed environmental sample and homogenizing the mixed environmental sample to provide an input sample for the application-centric biomining (see, e.g., FIG. 1).

In embodiments comprising use of a plurality of environmental samples to produce an input sample, collecting and mixing multiple environmental samples may serve to maximize not only the statistical sample space of microbes to screen from but also the combinations of microbes present in microbial consortia identified and/or produced using the technologies described herein that are applied to the input sample. Further, collecting and mixing multiple environmental samples to produce an input sample upon which the technologies described herein are applied may produce novel microbial consortia that do not exist in nature by combining microbes that normally do not live in the same environment in nature. In some embodiments, various environmental samples from geographically disparate areas may be mixed to further increase the statistical sample space of combinations of microbial consortia. For instance, embodiments provide that a plurality of environmental samples may be obtained wherein each environmental sample is taken from a different ecosystem, habitat, and/or ecological niche. Embodiments further provide that a plurality of environmental samples may be obtained from sites that are separated from each other by 1 m, 10 m, 100 m, 1000 m, 10,000 m, or by more than 10,000 m. In some embodiments, the samples are obtained from two or more points anywhere on the Earth, including above and below the surface of land and water areas of the Earth.

In some instances, multiple input samples 104 may be created during the collection. Each input sample of the multiple input samples 104 may comprise a different combination of individual environmental samples that are mixed together. For example, environmental samples A, B, and C (from one or more different ecosystems, habitats, and/or ecological niches) may be mixed to provide an input sample comprising A and B, B and C, or A and C. As a further example, environmental samples A, B, C, and D (from one or more different ecosystems, habitats, and/or ecological niches) may be mixed to provide an input sample comprising A, B, and C; A, B, and D; A, C, and D; or B, C, and D. As another example, environmental samples A, B, C, D, and E (from one or more different ecosystems, habitats, and/or ecological niches) may be mixed to provide an input sample comprising A and B; A and C; A and D; A and E; B and C; B and D; B and E; C and D; C and E; D and E; A, B, and C, A, B, and D; A, B, and E; A, C, and D; A, C, and E; A, D, and E; B, C, and D; B, C, and E; B, D, and E; C, D, and E; A, B, C, and D; A, B, C, and E; A, B, D, and E; A, C, D, and E; B, C, D, and E; or A, B, C, D, and E. Each input sample of the multiple input samples 104 may comprise a range of fractional compositions of any two individual environmental samples of a plurality of individual samples that are mixed together to provide the input sample. For example, any two individual environmental samples may be mixed together to provide an input sample comprising a fractional composition of a first environmental sample ranging from 0.01 to 0.99 (e.g., comprising 0.01, 0.05, 0.10, 0.20, 0.30, 0.40, 0.50, 0.60, 0.70, 0.80, 0.90, 0.95, or 0.99 of the first environmental sample) and comprising a fractional composition of a second environmental sample ranging from 0.99 to 0.01 (e.g., comprising 0.99, 0.95, 0.90, 0.80, 0.70, 0.60, 0.50, 0.40, 0.30, 0.20, 0.10, 0.05, or 0.01 of the second environmental sample).

The input sample 104 may be isolated and developed using variations of quantity and type environmental samples mixed. This is because it is recognized that a combination of microbes may not only be beneficial but may also cause individual microbes to become less effective or be dominated by microbes from foreign environmental samples. Further, embodiments of the technology comprise use of a single environmental sample that is homogenized to provide the input sample. One of ordinary skill in the art understands that a single environmental sample may comprise multiple individual ecosystems or ecological niches that are unmixed in nature but that become mixed when the single sample is homogenized. For example, an environmental sample may comprise a plurality of separate subsamples than are present as separate strata, layers, or subcommunities e.g., strata of a cylindrical soil core sample, strata of a microbial mat sample, strata of a water column sample, subcommunities of a microbial community comprising a biofilm, etc.

Thus, embodiments of the methods provided herein comprise use of a single environmental sample that is homogenized to provide an input sample 104 and/or comprise use of a plurality of environmental samples that are mixed and homogenized to provide an input sample 104.

In some embodiments, e.g., as shown in FIG. 1, a selection 106 of an input sample (e.g., an environmental sample or a mixed environmental sample of a plurality of mixed environmental samples) 104 based on one or more criteria 108 may be performed. In some instances, at least an initial focus may be on microbial consortia that are known in the environmental samples to fix carbon and nitrogen, such as the previously mentioned cyanobacteria and Azotobacter. The focus on a particular microbial consortium may be driven by the target species. In another example, if the target species is a legume, the focus may be on Gluconacetobacter and Herbaspirillium because they are more effective at fixing nitrogen in leaves and stems, on Azospirillium because it is also effective at fixing nitrogen in stems and roots, and on Azotobacter and Beijerinckia because they are effective at fixing nitrogen in the legume's rhizosphere.

A culture 110 of the input sample may be performed under one or more environmental conditions. In some instances, input samples may be stored in columns that admit light for photosynthesis. In some embodiments, the culture media are provided without nitrogen or carbon (e.g., nitrogen-free and carbon-free media or “C/N-free media”) that would interfere with the determination that microbial consortia were responsible for any measured nitrogen or carbon uptake. The input sample may be subject to nitrogen for fixation either by supplying nitrogen from the ambient concentration or by bubbling in anoxic N₂ and supplying salts and other nutrients known to be needed by the microbes to perform nitrogen fixation. The input samples may also be subjected to CO₂, e.g., either by ambient concentrations or via bubbling in CO₂.

After culturing and time, a testing 112 of the culture may be performed based on one or more variables 114. In some instances, climate change variables relevant to the desired biomining results are tested. In this case, the input samples may be tested for increased carbon and nitrogen or the ability of the resident microbiome to fix CO₂ and or nitrogen. Measurement may be by mass. The DNA of microbes that comprise candidate microbial consortia are then isolated and sequenced for identification. Within the DNA, for each microbe, a biomarker, such as 16S rRNA or GroEL is identified. These biomarkers will assist in future microbe identification. The cultures are then tested on nitrogen and carbon-free media in culture plates to measure survival time and/or persistence. Based at least on carbon capture, nitrogen fixation and/or persistence, selection 116 of one or more microbial cultures and/or specific portions of one or more microbial cultures is performed to provide cultures for testing 118.

In some cases, additives may be applied to encourage uptake of a microbial consortium by an environment (e.g., a soil) or culture medium. For example, microbial consortia may require carbon, energy, nitrogen, micronutrients, and reducing equivalents. As a specific example, a water and glucose spray can encourage E. coli in an environment or culture medium to generate reducing equivalents and 4-Carbon backbones used in CO₂ sequestration. By way of another example, organisms containing high levels of cellulases and/or lignases may be added to an environment or culture medium to aid the degradation of crop residue. The above process may be iterated several times through multiple iterations 120 and/or 122, with each iteration further isolating and generating identification information 124 for microbes and the specific microbial consortia that achieved the desired results on the selected variables, e.g., climate variables, carbon sequestration, nitrogen fixation, and survival time/persistence.

In some embodiments, the selection of microbes and microbial consortia to further test is aided with statistical models and computational methods including machine learning. Statistical models embodied in machine learning models may be used to direct the selection of microbes both in application-centric biomining as well as traditional biomining. For example, during experimentation and iteration, data around specific environmental sample source locations, environmental sample composition, microbes, and their associated genetic biomarkers, microbial consortia may be correlated with results on various variables, e.g., climate change or otherwise. In some instances where the machine learning model is to supplement traditional biomining, the data may also be supplemented with information capturing the phenotypes of microbes.

Upon achieving a critical mass of data 126, the data may be developed into a machine learning model 128 that correlates microbes and biomarkers, and microbe combinations to variables under test. For application-centric biomining, selection 130 of microbial consortia for initial testing, and/or selection of environmental sample characteristics may be suggested by the machine learning model 128 as results 132 based on the variables 134 under test. For traditional biomining, desired phenotypes can be input along with desired results 132 on variables 134 under test, and related microbes may be suggested by the machine learning model for further test.

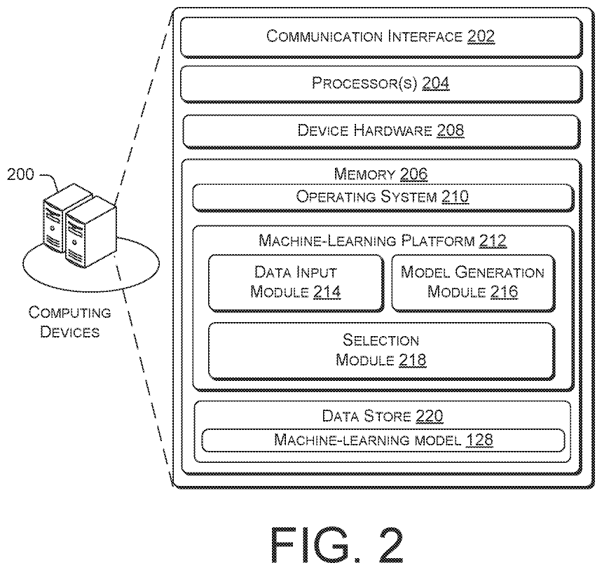

In some embodiments, e.g., as shown in FIG. 2, computing devices support machine learning techniques with respect to application-centric microorganism screening for effective variables and biomining. The computing devices 200 may provide a communication interface 202, one or more processors 204, memory 206, and device hardware 208. The communication interface 202 may include wireless and/or wired communication components that enable the devices to transmit data to and receive data from other networked devices. The device hardware 208 may include additional interface, data communication, or data storage hardware. For example, the hardware interfaces may include a data output device (e.g., visual display, audio speakers), and one or more data input devices. The data input devices may include, but are not limited to, combinations of one or more of keypads, keyboards, mouse devices, touch screens that accept gestures, microphones, voice or speech recognition devices, and any other suitable devices.

The memory 206 may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device. The computing devices 200 may also be in the form of either virtual machines or containers on virtual machines, such as provided via Kubernetes or Docker. In this case, virtual machines are hosted on a physical computer platform and served via a hypervisor. Colloquially, virtual machine configurations may be referred to as “the Cloud.”

The processors 204 and the memory 206 of the computing devices 200 may implement an operating system 210. In turn, the operating system 210 may provide an execution environment for the machine learning platform 212. The operating system 210 may include components that enable the computing devices 200 to receive and transmit data via various interfaces (e.g., user controls, communication interface, and/or memory input/output devices), as well as process data using the processors 204 to generate output. The operating system 210 may include a presentation component that presents the output (e.g., display the data on an electronic display, store the data in memory, transmit the data to another electronic device, etc.). Additionally, the operating system 210 may include other components that perform various additional functions generally associated with an operating system.

The machine learning platform 212 may include a data input module 214, a model generation module 216, and a selection module 218. The modules may include routines, program instructions, objects, and/or data structures that perform particular tasks or implement particular abstract data types. The memory 206 may also include a data store 220 that is used by the machine learning platform 212.

The data input module 214 may receive data from various sources, such as databases or data that is inputted via a user interface. The data input module 214 may use data adaptors to retrieve data from the databases of the data sources. For example, the data input module 214 may use data-agnostic data adaptors to access unstructured databases, and/or database-specific data adaptors to access structured databases. The data received may include the identification information of microbes, such as DNA biomarkers, phenotype information, environmental variables (e.g., types of nutrients, CO₂ level, amount of sunlight, etc.), environmental sample characteristics (e.g., composition, source location, etc.), associated microbe growth information, and/or so forth.

The model generation module 216 may train a machine learning model, such as the machine learning model 128, via a model training algorithm. The model training algorithm may implement a training data input phase, a feature engineering phase, and a model generation phase. In the training data input phase, the model training algorithm may receive training data, such as the data received via the data input module 214. During the feature engineering phase, the model training algorithm may pinpoint features in the training data. Accordingly, feature engineering may be used by the model training algorithm to figure out the significant properties and relationships in the training data that aid a machine learning model to distinguish between different classes of data. During the model generation phase, the model training algorithm may select an initial type of machine learning algorithm to train a machine learning model using the training data. Following the application of a selected machine learning algorithm to the training data, the model training algorithm may determine a training error measurement of the machine learning model. If the training error measurement exceeds a training error threshold, the model training algorithm may use a rule engine to select a different type of machine learning algorithm based on a magnitude of the training error measurement. The different types of machine learning algorithms may include a Bayesian algorithm, a decision tree algorithm, a support vector machine (SVM) algorithm, an ensemble of trees algorithm (e.g., random forests and gradient-boosted trees), an artificial neural network, and/or so forth. The training process is generally repeated until the training results fall below the training error threshold, and the trained machine learning model is generated. The trained machine learning model 128 may be stored in the data store 220.

The selection module 218 may apply the trained machine learning model 128 to one or more query variable values to generate query results for biomining. In some instances, a selection of a microbial consortia for initiating testing and/or a selection of environmental sample characteristics may be suggested by the selection module 218 applying the machine learning model 128 to the query variable values. In other instances, a desired phenotype may be inputted along with a desired result (e.g., a desired measurement of CO₂ sequestration, a persistence time of CO₂ sequestration, and/or so forth) on variables (e.g., an amount of nitrogen fixation, a survival time of a microbe, and/or so forth) under test. In turn, the selection module 218 may apply the machine learning model 128 to the inputted data to suggest related microbes for further test.

Accordingly, with a statistically significant amount of data, machine learning models may be developed to assist with the selection of microbes and microbial consortia. If the machine learning model is supplemented with phenotype data for the constituent microbes, the machine learning model may also augment traditional biomining.

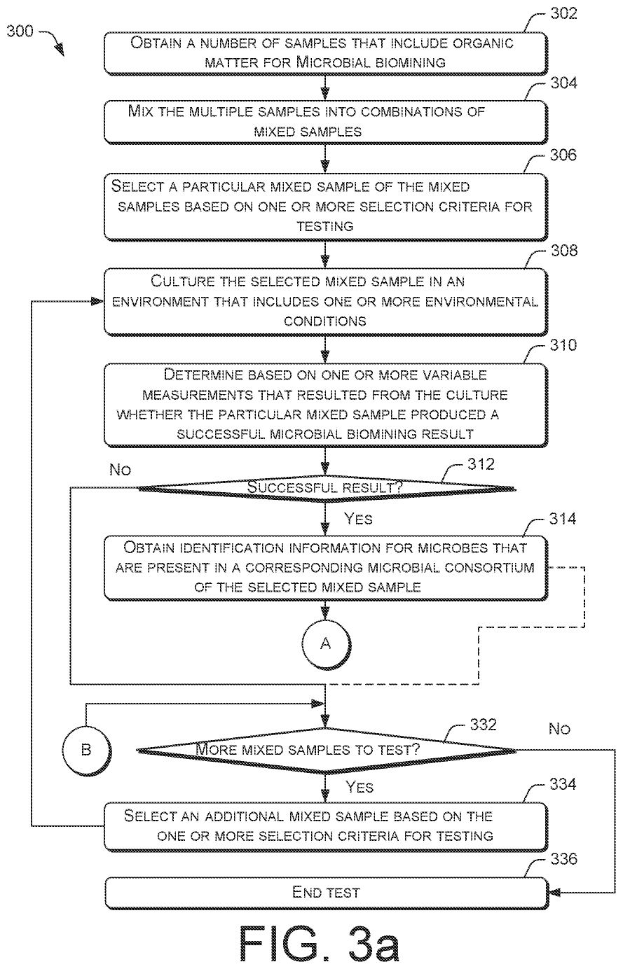

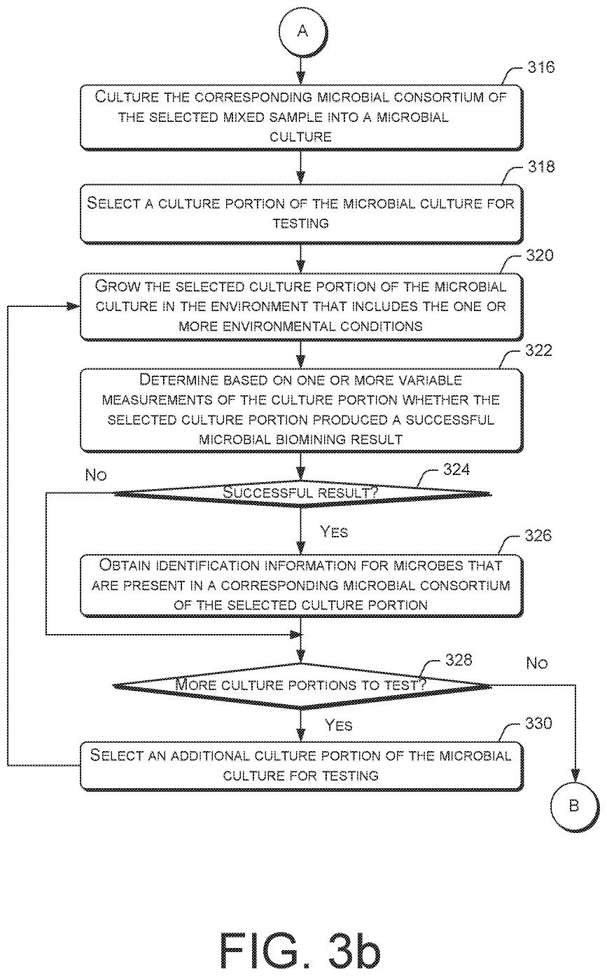

In some embodiments, e.g., as shown in FIGS. 3a and 3b, the technology provides a process (e.g., process 300) for performing an application-centric microorganism screening method. The order in which the operations are described in the example process 300 is not intended to be construed as a limitation, and any number of the described blocks can be combined in any order and/or in parallel to implement the process. Further, in some embodiments, the process 300 may be performed by obtaining a number of environmental samples 302 and mixing the environmental samples into combinations of mixed samples 304 to provide a number of input samples (e.g., mixed samples) for selection at step 306. In some embodiments, a single environmental sample may be homogenized to provide an input sample that is selected for input at step 306. In some embodiments, a number of single environmental samples and/or a number of mixed environmental samples may provide a plurality of input samples for selection at step 306. Accordingly, while the methods described herein and in FIGS. 3a and 3b are described in terms of obtaining a number of environmental samples 302 and mixing the multiple environmental samples 304, and selecting a mixed environmental sample 306 for culturing at step 308, the technology is not limited to methods comprising mixing multiple environmental samples and includes embodiments in which a single environmental sample is homogenized and provided as a selected sample for culturing at step 308. Further, embodiments comprise producing and providing a plurality of homogenized single environmental samples and selection a homogenized single environmental sample from the plurality of homogenized single environmental samples for culturing at step 308.

Thus, the steps (e.g., steps 302, 304, and 306) of a process (e.g., process 300) for performing an application-centric microorganism screening method is to be understood as comprising steps of providing a number (e.g., one or more) of mixed environmental samples produced my mixing and homogenizing multiple environmental samples or as comprising a step of providing a number (e.g., one or more) of single environmental samples that is/are homogenized for culturing at step 308. Reference to a mixed environmental sample throughout the description of the method is to be understood as referring to a mixed environmental sample produced by mixing and homogenizing multiple environmental samples or to a single environmental sample that is homogenized. Reference to multiple mixed environmental samples throughout the description of the method is to be understood as referring to a plurality of mixed environmental samples, wherein each mixed environmental sample of the plurality of mixed environmental samples is produced by mixing and homogenizing multiple environmental samples and/or is a single environmental sample that is homogenized.

In some embodiments, e.g., at block 302, multiple environmental samples that include organic matter may be obtained for biomining. At block 304, the multiple environmental samples may be mixed into combinations of mixed environmental samples. For example, the multiple environmental samples may be from different geographical areas so that the environmental samples contain different consortia of microbes. The mixing 304 may be performed by variation of quantity and type of environmental samples to serve to maximize microbe combinations. For example, environmental samples A, B, and C (from one or more different ecosystems, habitats, and/or ecological niches) may be mixed (e.g., at block 304) to provide an input sample comprising A and B, B and C, or A and C. As a further example, environmental samples A, B, C, and D (from one or more different ecosystems, habitats, and/or ecological niches) may be mixed (e.g., at block 304) to provide an input sample comprising A, B, and C; A, B, and D; A, C, and D; or B, C, and D. As another example, environmental samples A, B, C, D, and E (from one or more different ecosystems, habitats, and/or ecological niches) may be mixed (e.g., at block 304) to provide an input sample comprising A and B; A and C; A and D; A and E; B and C; B and D; B and E; C and D; C and E; D and E; A, B, and C; A, B, and D; A, B, and E; A, C, and D; A, C, and E; A, D, and E; B, C, and D; B, C, and E; B, D, and E; C, D, and E; A, B, C, and D; A, B, C, and E; A, B, D, and E; A, C, D, and E; B, C, D, and E; or A, B, C, D, and E. Each input sample of the multiple input samples may comprise a range of fractional compositions of any two individual environmental samples of a plurality of individual samples that are mixed together to provide the input sample. For example, any two individual environmental samples may be mixed together to provide an input sample comprising a fractional composition of a first environmental sample ranging from 0.01 to 0.99 (e.g., comprising 0.01, 0.05, 0.10, 0.20, 0.30, 0.40, 0.50, 0.60, 0.70, 0.80, 0.90, 0.95, or 0.99 of the first environmental sample) and comprising a fractional composition of a second environmental sample ranging from 0.99 to 0.01 (e.g., comprising 0.99, 0.95, 0.90, 0.80, 0.70, 0.60, 0.50, 0.40, 0.30, 0.20, 0.10, 0.05, or 0.01 of the second environmental sample).

At block 306, a particular mixed environmental sample of the mixed environmental samples may be selected based on one or more selection criteria for testing. For example, mixed environmental samples may be selected in some instances based on whether they at least contain certain microbial species and/or are demonstrate certain properties (e.g., functions), such as the ability to fix a certain amount of nitrogen or fix a certain quantity of carbon. At block 308, the selected mixed environmental sample may be cultured in an environment that includes one or more environmental conditions. For example, the environmental conditions may include a particular concentration of N₂ gas, a particular concentration of CO₂ gas, availability of one or more specific nutrients, one or more specific salts, one or more specific additives, etc., and/or so forth. The selected mixed environmental sample may be cultured in the environment for a predefined time period of minutes, hours, days, weeks, months, or years.

At block 310, a determination may be made based on one or more variable measurements that resulted from the culture of the selected mixed environmental sample whether the particular mixed environmental sample produced a successful biomining result. In various embodiments, the variables may include variables (e.g., climate change variables), such as an absolute amount of CO₂ sequestered by a biomass in the culture, a ratio of biomass to sequestered CO₂, an amount of time that CO₂ is sequestered by the biomass, and/or so forth. Accordingly, a successful biomining result may be determined when each variable measurement in a set of one or more variable measurements obtained for the culture of the selected mixed environmental sample at least met a corresponding variable measurement threshold. For example, the meeting of a measurement threshold may be the result of an increase in carbon sequestration, an increase in nitrogen fixation, an increase in biomass, or having a microbe that is able to meet a particular survival time. At decision block 312, if the culture of the selected mixed environmental sample produced a successful result (“yes” at decision block 312), the process 300 may proceed to block 314.

At block 314, identification information for the microbes that are present in a corresponding microbial consortium of the selected mixed environmental sample may be obtained. For example, the DNA of the microbes that comprise the corresponding microbial consortium may be isolated and sequenced (e.g., as described herein). Within the DNA for each microbe, a biomarker, such as 16S rRNA or GroEL, may be identified.

At block 316, the corresponding microbial consortium of the selected mixed environmental sample may be cultured into a microbial culture. At block 318, a culture portion of the microbial culture may be selected for testing. In some instances, the culture portion may be a randomly selected portion of the culture. In other instances, the culture portion may be selected based on whether the culture portion at least contains certain microbial species and/or is able to demonstrate certain properties (e.g., functions), such as the ability to fix a certain amount of nitrogen, fix a certain quantity of carbon, and/or have a certain survival time/persistence. At block 320, the selected culture portion of the microbial culture may be grown in an environment that includes one or more environmental conditions. For example, the environmental conditions may include a particular concentration of N₂ gas, a particular concentration of CO₂ gas, availability of one or more specific nutrients, one or more specific salts, one or more specific additives, etc., and/or so forth. The selected culture portion may be grown in the environment for a predetermined time period.

At block 322, a determination may be made based on one or more variable measurements of the selected culture portion whether the selected culture portion produced a successful microbial biomining result. In various embodiments, the variables may include variables (e.g., climate change variables), such as an absolute amount of CO₂ sequestered by a biomass of the selected culture portion, a ratio of biomass to sequestered CO₂, an amount of time that CO₂ is sequestered by the biomass, and/or so forth. Accordingly, a successful microbial biomining result may be determined when each variable measurement in a set of one or more variable measurements obtained for the culture portion at least met a corresponding variable measurement threshold. For example, the meeting of a measurement threshold may be the result of an increase in carbon sequestration, an increase in nitrogen fixation, an increase in biomass, or having a microbe that is able to meet a particular survival time.

At decision block 324, if the selected culture portion produced a successful biomining result (“yes” at decision block 324), the process 300 may proceed to block 326. At block 326, identification information for the microbes that are present in a corresponding microbial consortium of the selected culture portion may be obtained. For example, the DNA of the microbes that the corresponding microbial consortium may be isolated and sequenced. Within the DNA for each microbe, a biomarker, such as 16S rRNA or GroEL, may be identified.

Subsequently, the process 300 may proceed to decision block 328. Returning to decision block 324, if the selected culture portion did not produce a successful biomining result (“no” at decision block 324), the process 300 may proceed directly to decision block 328. At decision block 328, if there are more culture portions of the culture to test (“yes” at decision block 328), the process 300 may proceed to block 330. For example, there may be more culture portions to test if a number of culture portions of the microbial culture selected for testing has not yet reached a threshold test number, if a number of successful biomining results for the microbial culture has not yet reached a success threshold number, or if there is still a portion of the microbial culture remaining for testing. At block 330, an additional culture portion of the microbial culture may be selected for testing. Subsequently, the process 300 may return to block 320.

However, if there are no more culture portions of the culture to test (“no” at decision block 328), the process 300 may proceed to decision block 332. At decision block 332, if there are more mixed environmental samples to be tested (“yes” at decision block 332), the process 300 may proceed to block 334. At block 334, an additional mixed environmental sample may be selected based on one or more selection criteria for testing. However, if there are no more mixed environmental samples to be tested (“no” at decision block 332), the process 300 may terminate at block 334 such that the testing ends. For example, there may be more mixed environmental samples to test if a number of mixed environmental samples selected for testing has not yet reached a threshold test number, if a number of successful biomining results for the combinations of mixed environmental samples has not yet reached a success threshold number, or if there is still a mixed environmental sample remaining for testing.

Returning to decision block 312, if the culture of the selected mixed environmental sample did not produce a successful result (“no” at decision block 312), the process 300 may proceed directly to decision block 332. In some alternative embodiments, the process 300 may proceed directly from the block 314 to decision block 332 instead of proceeding through the blocks 316-330 prior to proceeding to decision block 332.

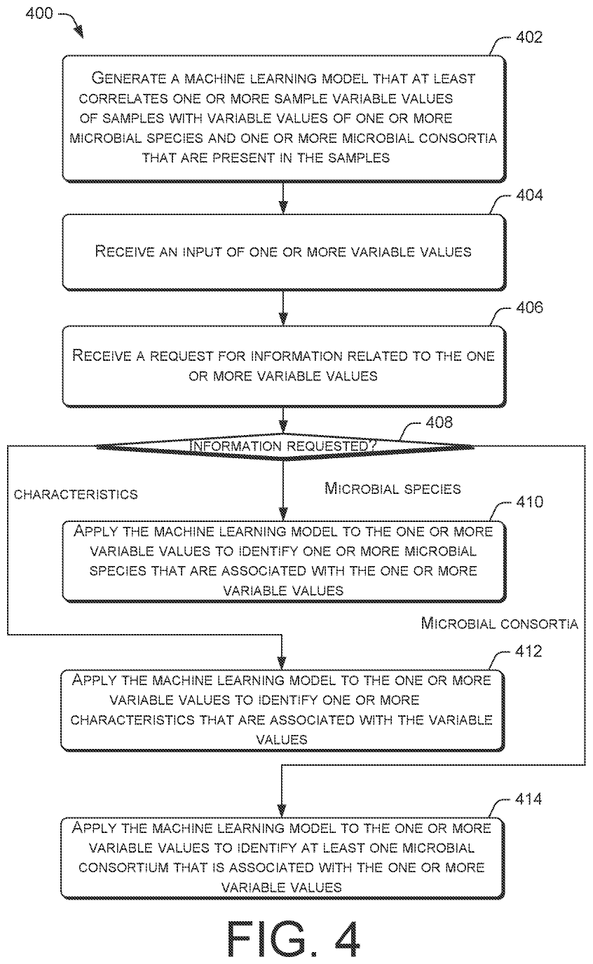

In some embodiments, e.g., as shown in FIG. 4 the technology provides machine learning techniques to identify microbial species and other information related to one or more variables. The example process 400 is illustrated as a collection of blocks in a logical flow chart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, code segments, programs, objects, components, data structures, and the like that perform particular functions or implement particular abstract data types. The order in which the operations are described is not intended to be construed as a limitation, and any number of the described blocks can be combined in any order and/or in parallel to implement the process.

At block 402, a machine learning platform may generate a machine learning model that at least correlates one or more environmental sample variable values of environmental samples with microbial variable values of one or more microbial species and one or more microbial consortia that are present in the one or more environmental samples. In various embodiments, the machine learning model may further correlate such variable values with values of environmental variables (e.g., climate change variables). For example, during experimentation and iterations of a microbial identification process, such as the process 300, data relating to specific environmental sample source locations, environmental sample composition, microbes, and their associated genetic biomarkers, microbial consortia may be correlated with results on various variables (e.g., climate change or otherwise) and used as training data for generating a machine-learning model. The variables otherwise correlated may include a mass ratio of biomass to the absolute amount fixed nitrogen, a total profit derived from CO₂ sequestration by the biomass, a ratio of food mass produced to mass of CO₂ sequestered by the biomass, an amount of time that CO₂ is sequestered by the biomass, and/or so forth.

At block 404, the machine learning platform may receive an input of one or more variable values. For example, the variable values may include phenotypes of microbes, a desired amount of nitrogen fixation, a desired amount of carbon sequestration, environmental sample characteristics, one or more climate change variables, and/or so forth. At block 406, the machine learning platform may include a request for information related to the one or more variable values. At decision block 408, if the information requested includes a microbial species that is related to the one or more variable values, the process 400 may proceed to block 410. At block 410, the machine learning platform may apply the machine learning model to the one or more variables to identify one or more microbial species that are associated with the one or more variable values. In some instances, a microbial species that is suggested by the machine learning model may be used for further testing for microbial biomining.

Returning to decision block 408, if the information requested includes environmental characteristics that are related to the one or more variables, the process 400 may proceed to block 412. At block 412, the machine learning platform may apply the machine learning model to the one or more variables to identify one or more environmental characteristics that are associated with the one or more variable values.

Returning to decision block 408, if the information requested includes microbial consortia that are related to the one or more variables, the process 400 may proceed to block 414. At block 414, the machine learning platform may apply the machine learning module to the one or more variable values to identify at least one microbial consortium that is associated with the one or more variable values. In some instances, a microbial consortium that is suggested by the machine learning model may be used for further testing for microbial biomining.

In contrast to conventional biomining, the application-centric biomining technology described herein starts with a large sample of microbes (e.g., from one or more environmental samples) and microbial consortia (e.g., comprising one or more microbes from a natural consortium and/or one or more microbes from different environments, ecosystems, habitats, and/or ecological niches) and produces new consortia comprising new combinations of microbes acting in concert. By testing for application-specific variables, microbes and microbial consortia providing the desired results may then be sequenced and then sub-cultured until the desired microbes and microbial consortia are identified and/or isolated. With a statistically significant amount of data, machine learning models may be developed to assist with the selection of microbes and microbial consortia. If the machine learning model is supplemented with phenotype data for the constituent microbes, the machine learning model may also augment traditional biomining.

As described herein, application-centric biomining focuses on the variables under test rather than the underlying phenotypes of the microbes. Variables under test may be the immediate variables of interest such as measures of carbon sequestration and of related dependent variables such as nitrogen fixation. Variables may be biological in nature, such as survival time and/or persistence of microbes. Because the variables under test are not tied to phenotypes or other directly to microbes, the variables under test can be global, such as the impact on global climate change and/or global food production. Furthermore, the variables under test need not be biological or chemical in nature. Variables under test may be economic such as profits from carbon sequestration, and comparisons of food production vs carbon sequestration.

In a particular example related to agriculture and soils, this decoupling of the variables under tests enables a wide range of a finer-grained analysis of agriculture. In the past, the impact of one farm could be differentiated by soil management techniques, and crop management techniques. However, application-specific biomining provides the ability to select a variable and find a microbial consortium to maximize the desired results. Since those results need not be agricultural variables, application-specific biomining increases the ability to bind agricultural performance and production to an arbitrary variable such as farm economics and climate change.

In some embodiments, the technology provides additional methods for selecting a microbial consortium that provides a specified function. In some embodiments, the technology provides a method for screening a microbial community, a microbial consortium and/or a plurality of microbes to produce and/or to identify a microbial consortium that provides a specified function. In some embodiments, the technology produces a microbial consortium not found in nature by combining microbes from different environments, ecological niches, and/or habitats (e.g., microbes that are not found together in nature).

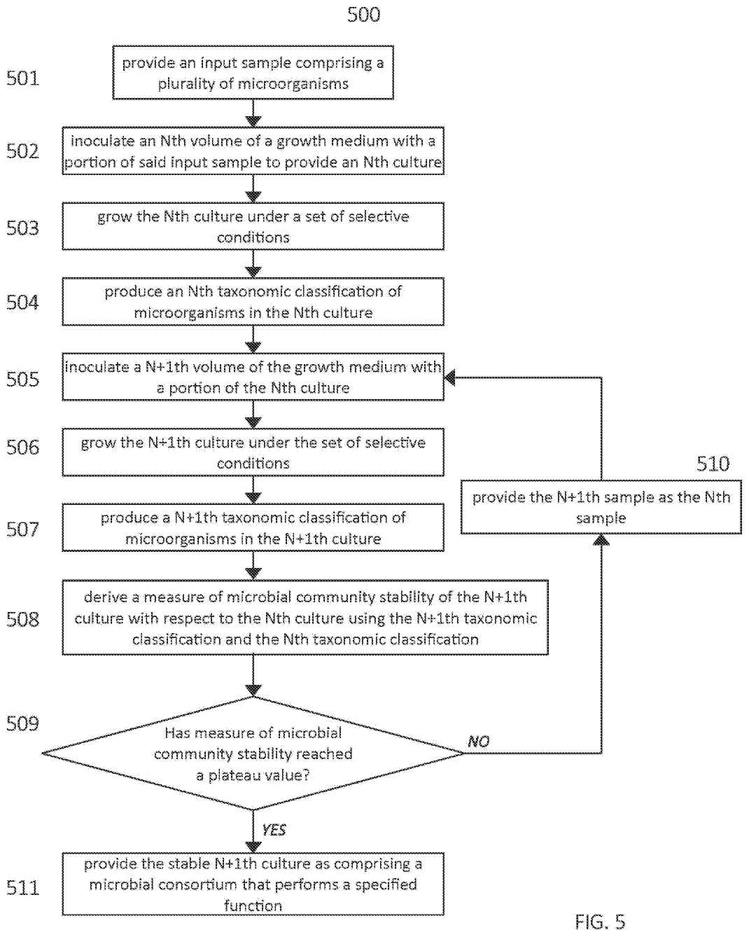

In some embodiments, e.g., as shown in FIG. 5, the technology provides methods for producing a microbial consortium that provides a specified function. Methods comprise providing (501) a sample comprising a plurality of microorganisms; inoculating (502) an Nth volume of a growth medium with a portion of the sample to provide an Nth culture; growing (503) the Nth culture under a set of selective conditions; producing (504) an Nth taxonomic classification of microorganisms in the Nth culture; inoculating (505) an N+1th volume of the growth medium with a portion of the Nth culture; growing (506) the N+1 culture under the set of selective conditions; producing (507) an N+1th taxonomic classification of microorganisms in the N+1th culture; and deriving (508) a measure of microbial community stability of the N+1th culture with respect to the Nth culture using the N+1th taxonomic classification and the Nth taxonomic classification. The measure of microbial community stability is monitored to identify that the measure of microbial community stability has reached a plateau value. If the measure of microbial community stability has not reached a plateau value (509), then steps 505-508 are performed again by providing (510) the N+1th sample as the Nth sample at step 505. If the measure of microbial community stability has reached a plateau value (509), the method comprising providing (511) the stable N+1th culture as a culture comprising a microbial consortium that performs a specified function. In some embodiments, steps 505-508 are repeated 2, 3, 4, 5, 6, 7, 8, 9, or 10 or more times.

In some embodiments, methods further comprise isolating each of the microorganisms of the stable microbial consortium in a pure culture. In some embodiments, methods further comprise obtaining a genome sequence of each of the microorganisms of the stable microbial consortium in a pure culture. In some embodiments, methods further comprise storing the stable microbial consortium and/or each of the microorganisms of the stable microbial consortium (e.g., by freezing (e.g., at −80 C)). In some embodiments, methods further comprise measuring the specified function of the stable microbial consortium using test substrates and methods of measuring the output of the function.

The technology is not limited in the types of samples comprising microorganisms (e.g., environmental samples) that are used as starting material (e.g., an input sample) upon which the methods (e.g., methods for selecting a microbial consortium and/or methods for screening to identify a microbial consortium) as described herein are performed. In some embodiments, the input sample used can be an environmental sample from any source, for example, naturally occurring or artificial atmosphere, water systems and sources, soil or any other sample of interest. In some embodiments, the environmental sample may be obtained from, for example, indoor or outdoor air or atmospheric particle collection systems; indoor surfaces and surfaces of machines, devices, or instruments. In some embodiments, ecosystems are sampled (e.g., in some embodiments, a sample is an environmental sample taken from an ecosystem). Ecosystems can be terrestrial and include all known terrestrial environments including, but not limited to soil, surface, and above surface environments. Ecosystems include those classified in the Land Cover Classification System (LCCS) of the Food and Agriculture Organization and the Forest-Range Environmental Study Ecosystems (FRES) developed by the United States Forest Service. Exemplary ecosystems include forests such as tropical rainforests, temperate rainforest, temperate hardwood forests, boreal forests, taiga, and montane coniferous forests; grasslands including savannas and steppes; deserts; wetlands including marshes, swamps, bogs, estuaries, and sloughs; riparian ecosystems, alpine, and tundra ecosystems. Ecosystems further include those associated with aquatic environments such as lakes, streams, springs, coral reefs, beaches, estuaries, sea mounts, trenches, and intertidal zones. Ecosystems also comprise soils, humus, mineral soils, and aquifers. Ecosystems further encompass underground environments, such as mines, oil fields, caves, faults and fracture zones, geothermal zones, and aquifers. Ecosystems additionally include the microbiomes associated with plants, animals, and humans. Exemplary plant associated microbiomes include those found in or near roots, bark, trunks, leaves, and flowers. Animal and human associated microbiomes include those found in the gastrointestinal tract, respiratory system, nares, urogenital tract, mammary glands, oral cavity, auditory canal, feces, urine, and skin. In some embodiments, the sample can be any kind of clinical or medical sample. For example, samples may be from blood, urine, feces, nares, the lungs, or the gut of mammals.

For instance, in some embodiments, one or more environmental samples are collected. If a single environmental sample is collected, methods comprise homogenizing the environmental sample to provide an input sample (e.g., at block 501). If a plurality of environmental samples is collected, methods comprise mixing the plurality of environmental samples to provide a mixed environmental sample and homogenizing the mixed environmental sample to provide an input sample (e.g., at block 501).

In embodiments comprising use of a plurality of environmental samples to produce an input sample, collecting and mixing multiple environmental samples may serve to maximize not only the statistical sample space of microbes to screen from but also the combinations of microbes present in microbial consortia identified and/or produced using the technologies described herein that are applied to the input sample. Further, collecting and mixing multiple environmental samples to produce an input sample upon which the technologies described herein are applied may produce novel microbial consortia that do not exist in nature by combining microbes that normally do not live in the same environment in nature. In some embodiments, various environmental samples from geographically disparate areas may be mixed to further increase the statistical sample space of combinations of microbial consortia. For instance, embodiments provide that a plurality of environmental samples may be obtained wherein each environmental sample is taken from a different ecosystem, habitat, and/or ecological niche. Embodiments further provide that a plurality of environmental samples may be obtained from sites that are separated from each other by 1 m, 10 m, 100 m, 1000 m, 10,000 m, or by more than 10,000 m. In some embodiments, the samples are obtained from two or more points anywhere on the Earth, including above and below the surface of land and water areas of the Earth.

In some instances, multiple input samples may be created during the collection. Each input sample of the multiple input samples may comprise a different combination of individual environmental samples that are mixed together. For example, environmental samples A, B, and C (from one or more different ecosystems, habitats, and/or ecological niches) may be mixed to provide an input sample comprising A and B, B and C, or A and C. As a further example, environmental samples A, B, C, and D (from one or more different ecosystems, habitats, and/or ecological niches) may be mixed to provide an input sample comprising A, B, and C; A, B, and D; A, C, and D; or B, C, and D. As another example, environmental samples A, B, C, D, and E (from one or more different ecosystems, habitats, and/or ecological niches) may be mixed to provide an input sample comprising A and B; A and C; A and D; A and E; B and C; B and D; B and E; C and D; C and E; D and E; A, B, and C, A, B, and D; A, B, and E; A, C, and D; A, C, and E; A, D, and E; B, C, and D; B, C, and E; B, D, and E; C, D, and E; A, B, C, and D; A, B, C, and E; A, B, D, and E; A, C, D, and E; B, C, D, and E; or A, B, C, D, and E. Each input sample of the multiple input samples may comprise a range of fractional compositions of any two individual environmental samples of a plurality of individual samples that are mixed together to provide the input sample. For example, any two individual environmental samples may be mixed together to provide an input sample comprising a fractional composition of a first environmental sample ranging from 0.01 to 0.99 (e.g., comprising 0.01, 0.05, 0.10, 0.20, 0.30, 0.40, 0.50, 0.60, 0.70, 0.80, 0.90, 0.95, or 0.99 of the first environmental sample) and comprising a fractional composition of a second environmental sample ranging from 0.99 to 0.01 (e.g., comprising 0.99, 0.95, 0.90, 0.80, 0.70, 0.60, 0.50, 0.40, 0.30, 0.20, 0.10, 0.05, or 0.01 of the second environmental sample).

The input sample may be isolated and developed using variations of quantity and type environmental samples mixed. This is because it is recognized that a combination of microbes may not only be beneficial but may also cause individual microbes to become less effective or be dominated by microbes from foreign environmental samples. Further, embodiments of the technology comprise use of a single environmental sample that is homogenized to provide the input sample. One of ordinary skill in the art understands that a single environmental sample may comprise multiple individual ecosystems or ecological niches that are unmixed in nature but that become mixed when the single sample is homogenized. For example, an environmental sample may comprise a plurality of separate subsamples than are present as separate strata, layers, or subcommunities e.g., strata of a cylindrical soil core sample, strata of a microbial mat sample, strata of a water column sample, subcommunities of a microbial community comprising a biofilm, etc.

Thus, embodiments of the methods provided herein comprise use of a single environmental sample that is homogenized to provide an input sample at step 501 and/or comprise use of a plurality of environmental samples that are mixed and homogenized to provide an input sample at step 501.

The technology provides methods for reduce the complexity of a community of microbes (e.g., present in an environmental sample) while selecting for a microbial consortium that performs a specified function and/or identifying a microbial consortium that performs a specified function. Exemplary functions for which microbial consortia may be selected and/or identified include, e.g., biodegradation, fermentation, production of chemical precursors, biosensing, nitrogen fixation, and carbon fixation.

In some embodiments, environmental samples are used to inoculate a culture medium and the inoculated culture medium is grown under selective conditions provided by the culture medium (e.g., presence, absence, or type of carbon source; presence, absence, or type of nitrogen source; presence, absence, or type of cofactors, minerals, vitamins, or other nutrients; presence, absence, or type of cations and/or anions; presence, absence, or type of trace minerals, cations, and/or anions; presence, absence, or type of a solid growth substrate such as sand or other solid substrate) or by selective conditions provided external to the growth medium (e.g., temperature; humidity; presence, absence, wavelength, and/or intensity of light; light/dark cycle; pressure; culture volume; culture volume material, size, or geometry; presence, absence, type, or strength of culture agitation; presence, absence, and/or type of gases provided).

In some embodiments, a culture is inoculated (e.g., at step 502 and/or step 505) and grown (e.g., at step 503 and/or step 506) for a length of time, e.g., 30 to 60 minutes (e.g., 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, or 60.0 minutes); 1 to 24 hours (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, or 24.0 hours); 1 to 30 days (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 days); and/or 1 to 10 weeks (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 weeks).

In some embodiments, empirical measurements of growth rate, time to exponential growth phase, time to culture saturation, or other culture growth characteristics are measured to identify a length of time for culture growth. In some embodiments, a growth time is selected that provides a culture at or near the end of exponential growth phase to provide a culture with a robust type and number of microorganisms for further characterization and/or selection. In some embodiments, growth is measured quantitatively and/or qualitatively using a measurement of the absolute or relative number of microorganisms in a defined volume of culture. In some embodiments, the absolute or relative number of microorganisms in a defined volume of culture is measured using light scattering, measuring dry or wet mass of solids (e.g., cells) isolated from the culture, counting colonies grown on solid medium using a portion of the culture, or measuring some other characteristic of the culture or a portion thereof that has a correlative or causal connection with the number of microorganisms in the culture. In some embodiments, growth is characterized by determining a growth curve; in some embodiments, growth is characterized by determining a doubling time and/or time to half saturation. In some embodiments, growth rates are modeled using empirical data (e.g., using a logarithmic model of growth).

In some embodiments, the microorganisms in a culture are characterized by shotgun metagenomic sequencing (e.g., at step 507). Techniques and systems to obtain genetic sequences from multiple organisms in a sample, such as an environmental or clinical sample, are well known by persons skilled in the art. For example, Zhou et al. (Appl. Environ. Microbiol. (1996) 62:316-322) provides a robust nucleic acid extraction and purification. This protocol may also be modified depending on the experimental goals and environmental sample type, such as soils, sediments, and groundwater. Many commercially available DNA extraction and purification kits can also be used. Samples with lower than 2 pg purified DNA may require amplification, which can be performed using conventional techniques known in the art, such as a whole community genome amplification (WCGA) method (Wu et al., Appl. Environ. Microbiol. (2006) 72, 4931-4941). Techniques and systems for obtaining purified RNA from environmental samples are also well known by persons skilled in the art. For example, the approach described by Hurt et al. (Appl. Environ. Microbiol. (2001) 67:4495-4503) can be used. This method can isolate DNA and RNA simultaneously within the same sample. A gel electrophoresis method can also be used to isolate community RNA (McGrath et al., J. Microbiol. Methods (2008) 75:172-176). Samples with lower than 5 pg purified RNA may require amplification, which can be performed using conventional techniques known in the art, such as a whole community RNA amplification approach (WCRA) (Gao et al., Appl. Environ. Microbiol. (2007) 73:563-571) to obtain cDNA. In some embodiments, environmental sampling and DNA extraction are conducted as previously described (DeSantis et al., Microbial Ecology, 53(3)371-383, 2007).

Isolated nucleic acids (e.g., metagenomic DNA) can be subject to a sequencing method to obtain metagenomic sequencing data. Sequencing methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively. Accordingly, metagenomic shotgun sequencing comprises, in some embodiments, pyrosequencing, sequencing-by-ligation, single molecule sequencing, sequence-by-synthesis (SBS), semiconductor sequencing, nanopore sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

Specific descriptions of some DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety); automated sequencing techniques; parallel sequencing of partitioned amplicons (PCT Publication No: WO2006084132 to Kevin McKernan et al., herein incorporated by reference in its entirety); and sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional descriptions of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety). See also, e.g., Voelkerding et al., Clinical Chem., 55: 641-658, 2009; MacLean et al., Nature Rev. Microbiol., 7: 287-296; each herein incorporated by reference in its entirety).

Metagenomic nucleotide sequence data can be analyzed to characterize the microbial community (e.g., microbial consortium) from which the metagenomic nucleic acids were obtained (e.g., at step 507). For example, in some embodiments, taxonomic units in a microbial community are taxonomically classified and/or identified by obtaining metagenomic nucleotide sequence data from the microbial community and using an algorithm that associates short genomic substrings (k-mers) in the metagenomic nucleotide sequence data with lowest common ancestor (LCA) taxa (e.g., using a curated database). See, e.g., e.g., Wood (2014) “Kraken: ultrafast metagenomic sequence classification using exact alignments” Genome Biology 15: R46 and Wood (2019) “Improved metagenomic analysis with Kraken 2” Genome Biology 20:257, each of which is incorporated herein by reference. In some embodiments, BLAST is used to identify the microbial taxonomic units present in a microbial community (e.g., microbial consortium). See, e.g., Altschul (1990) “Basic local alignment search tool” J Mol Biol 215:403-410, incorporated herein by reference. Other tools for identifying taxonomic units in a microbial community using metagenomic sequence data from the microbial community include, e.g., MEGAN (see, e.g., Huson (2007) “MEGAN analysis of metagenomic data” Genome Res 17:377-386, incorporated herein by reference); PhymmBL (see, e.g., Brady (2009) “Phymm and PhymmBL: metagenomic phylogenetic classification with interpolated Markov models” Nat Methods 6:673-676; and Brady (2011) “PhymmBL expanded: confidence scores, custom databases, parallelization and more” Nat Methods 8:367, each of which is incorporated herein by reference); and the Naïve Bayes Classifier (NBC) (see, e.g., Rosen (2008) “Metagenome fragment classification using N-mer frequency profiles” Adv Bioinformatics 2008:1-12, incorporated herein by reference).

In some embodiments, characterizing a microbial community comprises identifying the taxonomic units (e.g., strains, sub-species, species, genera, families) of organisms present in the microbial community in absolute and/or relative terms. In some embodiments, characterizing a microbial community comprises identifying the taxonomic units (e.g., strains, sub-species, species, genera, families) of organisms that have been enriched in a particular passage with respect to a previous passage or initial environmental sample, e.g., in relative terms.

In some embodiments, the technology provides an iterative method (e.g., method 500 comprising iterations of steps 505 to 510) in which a portion of a first culture is used to inoculate a second volume of fresh medium. Accordingly, in some embodiments, a portion of a first culture (e.g., a culture produced by inoculating a selective growth medium with an environmental sample) is used to inoculate a second culture (e.g., comprising the same or different growth medium as in the first sample). In some embodiments, a portion of a second culture is used to inoculate a third culture. In some embodiments, a portion of a third culture is used to inoculate a fourth culture. In some embodiments, a portion of a fourth culture is used to inoculate a fifth culture. In some embodiments, a portion of a fifth culture is used to inoculate a sixth culture. In some embodiments, a portion of a sixth culture is used to inoculate a seventh culture. In some embodiments, a portion of a seventh culture is used to inoculate an eighth culture. In some embodiments, a portion of an Nth culture is used to inoculate an N+1th culture. In some embodiments, the Nth culture is a first culture inoculated using at least a portion of an environmental sample. In some embodiments, the Nth culture is a second, third, fourth, fifth, sixth, seventh, eighth, etc. culture inoculated using at least a portion of a culture inoculated using a predecessor culture (e.g., a first, second, third, fourth, fifth, sixth, or seventh culture, respectively). As used herein, the process of iterative culturing by using a portion of an Nth culture to inoculate an N+1th culture is called “passaging” of the culture.

Further, a culture inoculated directly from an environmental sample may be referenced herein as a P0 (zero) culture; the first passage comprises using a portion of the P0 culture to inoculate fresh culture medium to produce a P1 culture; the second passage comprises using a portion of the P1 culture to inoculate fresh culture medium to produce a P2 culture; the third passage comprises using a portion of the P2 culture to inoculate fresh culture medium to produce a P3 culture; the fourth passage comprises using a portion of the P3 culture to inoculate fresh culture medium to produce a P4 culture; the fifth passage comprises using a portion of the P4 culture to inoculate fresh culture medium to produce a P5 culture; the sixth passage comprises using a portion of the P5 culture to inoculate fresh culture medium to produce a P6 culture; the seventh passage comprises using a portion of the P6 culture to inoculate fresh culture medium to produce a P7 culture; the eighth passage comprises using a portion of the P7 culture to inoculate fresh culture medium to produce a P8 culture; and the Nth passage comprises using a portion of the P(N−1) culture to produce a PN culture. As used herein, the term “passage number” refers a specific passaging as indicated by the number, e.g., passage number 1 refers to the first passage, passage number 2 refers to the second passage, etc.

In some embodiments, the volume of a portion of an Nth (e.g., first) culture used to inoculate an N+1th (e.g., second) culture) is from 100 μl to 100 L or more, depending on the scale of the culturing process (e.g., from research scale to a pilot scale to a commercial production scale). Accordingly, embodiments provide removing a volume of 100 μl to 100 L (e.g., 100, 150, 200, 250, 300, 350, 400, 450, 500, 550, 600, 650, 700, 750, 800, 850, 900, 950, or 1000 μl; 1, 2, 5, 10, 20, 50, 100, 150, 200, 250, 300, 350, 400, 450, 500, 550, 600, 650, 700, 750, 800, 850, 900, 950, or 1000 mL; or 1, 2, 5, 10, 20, 50, or 100 L) from one culture and adding the volume to fresh culture medium. In some embodiments, the ratio of the inoculating volume to the volume of fresh culture medium is from approximately 1:10 to 1:1000. Accordingly, in some embodiments, the volume of the fresh culture medium is from 1 ml to 100,000 L (e.g., 1; 2; 5; 10; 20; 50; 100; 200; 500; or 1000 mL; 1; 2; 5; 10; 20; 50; 100; 200; 500; 1000; 2000; 5000; 10,000; 20,000; 50,000; or 100,000 L).

In some embodiments, the stability of a microbial community and/or microbial consortium is measured (e.g., at step 508), e.g., by deriving a measure of similarity (or dissimilarity) between a first culture and a second culture inoculated using a portion of the first culture and, optionally, following the measure of similarity as a function of subsequent inoculations. In some embodiments, taxonomic classification and/or identification of the organisms in the microbial community (e.g., as provided by the taxonomic classifiers described above (e.g., Kraken 2)) can provide input into such measures of stability. In some embodiments, functional capabilities or functions provided by and/or present in the microbial community (e.g., genes, gene products, functional capabilities and/or activities) provide input into a measure of stability.

Various measures can be used to compare the similarities (or dissimilarities) of microbial communities, including estimates of the richness and diversity of a microbial community (see, e.g., Hughes (2001) “Counting the uncountable: statistical approaches to estimating microbial diversity” Appl. Environ. Microbiol. 67:4399-4406; and Ley (2005) “Obesity alters gut microbial ecology” Proc. Natl. Acad. Sci. USA 102:11070-11075, each of which is incorporated herein by reference) and estimates of alpha or beta diversity, e.g., the Bray-Curtis Dissimilarity Index (Bray and Curtis (1957) “An Ordination of the Upland Forest Communities of Southern Wisconsin” Ecol. Monogr. 27: 325-349, incorporated herein by reference). Bray-Curtis distances may be calculated using the bcdist function in the ecodist package (Goslee (2007) “The ecodist package for dissimilarity-based analysis of ecological data” J Stat Softw 22: 1-19, incorporated herein by reference). Correlation between Bray-Curtis distance matrices of community data, geographical distance, and environmental variables may be calculated using the mantel function in the vegan package (Oksanen, vegan: Community Ecology Package for R); see, e.g., Legendre, P. and Legendre, L. (2012) Numerical Ecology. 3rd English Edition. Elsevier, incorporated herein by reference).

Several tools are available that provide these and other estimates of microbial community structures (e.g., describing the abundance of community members). See, e.g., LIBSHUFF (Schloss (2004) “Integration of microbial ecology and statistics: a test to compare gene libraries” Appl. Environ. Microbiol. 70:5485-5492; and Singleton (2001) “Quantitative comparisons of 16S rRNA gene sequence libraries from environmental samples” Appl. Environ. Microbiol. 67:4374-4376, each of which is incorporated herein by reference), TreeClimber (Martin (2002) “Phylogenetic approaches for describing and comparing the diversity of microbial communities” Appl. Environ. Microbiol. 68:3673-3682; and Schloss (2006) “Introducing TreeClimber, a test to compare microbial community structures” Appl. Environ. Microbiol. 72:2379-2384, each of which is incorporated herein by reference), UniFrac (Lozupone (2005) “UniFrac: a new phylogenetic method for comparing microbial communities” Appl. Environ. Microbiol. 71:8228-8235, incorporated herein by reference), and analysis of molecular variance (AMOVA) (Excoffier (1992) “Analysis of molecular variance inferred from metric distances among DNA haplotypes: application to human mitochondrial DNA restriction data” Genetics 131:479-491; and Martin (2002) “Phylogenetic approaches for describing and comparing the diversity of microbial communities” Appl. Environ. Microbiol. 68:3673-3682, each of which is incorporated herein by reference); DOTUR (Schloss (2005) “Introducing DOTUR, a computer program for defining operational taxonomic units and estimating species richness” Appl. Environ. Microbiol. 71:1501-1506, incorporated herein by reference); and SONS (Schloss (2006) “Introducing SONS, a Tool for Operational Taxonomic Unit-Based Comparisons of Microbial Community Memberships and Structures” Appl Environ Microbiol. 726773-6779, incorporated herein by reference), which provides several measures including measures of membership (e.g., incidence-based Sorenson similarity index), community structure using abundance (e.g., Clayton θ (see, e.g., Yue (2001) “A nonparametric estimator of species overlap” Biometrics 57:743-9, incorporated herein by reference), and community richness (see, e.g., Chao (1984) “Non-parametric estimation of the number of classes in a population” Scand. J. Stat. 11:265-270; Chao (2005) “A new statistical approach for assessing similarity of species composition with incidence and abundance data” Ecol. Lett. 8:148-159; Chao (2000) “Estimating the number of shared species in two communities” Stat. Sinica 10:227-246; Chao (1992) “Estimating the number of classes via sample coverage” J. Am. Stat. Assoc. 87:210-217; and Chao (2006) “The applications of Laplace's boundary-mode approximations to estimate species richness and shared species richness” Aust. N. Z. J. Stat. 48:117-128, each of which is incorporated herein by reference).

As used herein, the term “stable”, when used in reference to a microbial community (e.g., a microbial community, a microbial consortium, a microbial culture, or other group, set, or collection of microorganisms), refers to a microbial community that does not significantly change (e.g., as measured by a measurement of similarity discussed above) from a first culture to a second culture when a portion of the first culture is used to inoculate a culture medium to produce the second culture when culture conditions, including external factors (light, nutrients, temperature, aeration, etc.), are the same for the first and second cultures. Accordingly, as used herein, the term “stability”, when used in reference to a microbial community (e.g., “microbial community stability”), refers to a qualitative or quantitative indicator or measurement of the change in a microbial community (e.g., a microbial community, a microbial consortium, a microbial culture, or other group, set, or collection of microorganisms) (e.g., as measured by a measurement of similarity discussed above) from a first culture to a second culture when a portion of the first culture is used to inoculate a culture medium to produce the second culture when culture conditions, including external factors (light, nutrients, temperature, aeration, etc.), are the same for the first and second cultures.

Thus, monitoring a similarity measurement of a culture, microbial community, and/or microbial consortium as a function of passage number (e.g., in steps 508, 509, and 511) provides a measurement of the stability of the culture, microbial community, and/or microbial consortium in the culture from the passaging process. A decrease in the rate of change of the similarity measurement as a function of passage number indicates an increase in the stability of the culture, microbial community, and/or microbial consortium. A plateauing or stabilization of the similarity measurement as a function of the passage number indicates that the culture, microbial community, and/or microbial consortium is at or approaching maximum stability (e.g., at step 509 and 511). For instance, in some embodiments, a plateau in the stability measure is reached when the stability measure is within 10 to 20% (e.g., 10, 11, 12, 13, 14, 15, 16, 17, 18, 19, or 20%) of the previous stability measure. In some embodiments, a plateau in the stability measure is reached when the stability measure is within 10 to 20% (e.g., 10, 11, 12, 13, 14, 15, 16, 17, 18, 19, or 20%) of the previous stability measure for a plurality of passagings (e.g., for 2, 3, 4, 5, 6, 7, or 8 passagings). In some embodiments, a plateau is the stability measure is reached when the slope of a line fitting the stability measure as a function of passage number is zero, substantially zero, or effectively zero.

Additionally, as used herein, the term “stable”, when used in reference to one or more functions provided and/or performed by a microbial community (e.g., a microbial community, a microbial consortium, a microbial culture, or other group, set, or collection of microorganisms), refers to one or more functions that do not significantly change (e.g., as measured by examination of metagenomic sequence and/or by inferring functions therefrom) from a first culture to a second culture when a portion of the first culture is used to inoculate a culture medium to produce the second culture when culture conditions, including external factors (light, nutrients, temperature, aeration, etc.), are the same for the first and second cultures. Accordingly, as used herein, the term “stability”, when used in reference to one or more functions provided by a microbial community (e.g., “functional stability”), refers to a qualitative or quantitative indicator or measurement of the change in one or more functions provided by a microbial community (e.g., a microbial community, a microbial consortium, a microbial culture, or other group, set, or collection of microorganisms) (e.g., as measured by a measurement of similarity discussed above) from a first culture to a second culture when a portion of the first culture is used to inoculate a culture medium to produce the second culture when culture conditions, including external factors (light, nutrients, temperature, aeration, etc.), are the same for the first and second cultures. Accordingly, functional stability and microbial stability may be independent such that a microbial community may be functionally stable but have changing membership and/or abundance of members such that the microbial community does not have microbial community stability. Thus, a microbial community may have both functional stability and microbial community stability; a microbial community may have neither functional stability nor microbial community stability; a microbial community may have functional stability (e.g., regardless of the state of microbial community stability); a microbial community may have microbial community stability (e.g., regardless of the state of functional stability).

Although the disclosure herein refers to certain illustrated embodiments, it is to be understood that these embodiments are presented by way of example and not by way of limitation. All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method, comprising:
obtaining multiple environmental samples that include organic matter for microbial biomining;
mixing the multiple environmental samples into combinations of mixed environmental samples;
selecting a particular mixed environmental sample of the mixed environmental samples based on one or more selection criteria for testing;
culturing the particular mixed environmental sample as selected in an environment that includes one or more environmental conditions; and
in response to determining based on one or more variable measurements that resulted from the culturing that the particular mixed environmental sample produced a successful microbial biomining result, obtaining identification information for microbes that are present in a corresponding microbial consortium of the particular mixed environmental sample.

2. The method of claim 1, further comprising in response to determining based on the one or more variable measurements that resulted from the culturing that the particular mixed environmental sample produced an unsuccessful microbial biomining result, selecting an additional mixed environmental sample based on the one or more selection criteria for testing.

3. The method of claim 1, further comprising:
selecting an additional mixed environmental sample of the mixed environmental samples based on the one or more selection criteria for testing;
culturing the additional mixed environmental sample as selected in an environment that includes one or more environmental conditions; and
in response to determining based on one or more variable measurements that resulted from the culturing that the additional mixed environmental sample produced an additional successful microbial biomining result, obtaining additional identification information for additional microbes that are present in an additional corresponding microbial consortium of the additional mixed environmental sample.

4. The method of claim 1, further comprising:
culturing the corresponding microbial consortium of the particular mixed environmental sample into a microbial culture;
growing a selected culture portion of the microbial culture in the environment that includes one or more environmental conditions; and
in response to determining based on one or more variable measurements of the selected culture portion that the selected culture portion produced a successful microbial biomining result, obtaining additional identification information for additional microbes that are present in an additional corresponding microbial consortium of the selected culture portion.

5. The method of claim 4, further comprising in response to determining based on one or more variable measurements of the selected culture portion that the culture portion produced an unsuccessful microbial biomining result, selecting an additional culture portion of the microbial culture for testing.

6. The method of claim 4, further comprising:
growing an additional selected culture portion of the microbial culture in the environment that includes one or more environmental conditions; and
in response to determining based on one or more variable measurements of the additional selected culture portion that the selected culture portion produced a successful microbial biomining result, obtaining further identification information for further microbes that are present in a further corresponding microbial consortium of the additional selected culture portion.

7. The method of claim 4, further comprising generating a machine learning model based on training data that includes the identification information and the additional identification information.

8. The method of claim 7, wherein the machine learning model at least correlates one or more environmental sample variable values of the multiple environmental samples with microbial variable values of one or more microbial species and one or more microbial consortia that are present in the multiple environmental samples.

9. The method of claim 8, further comprising:
receiving a request for information related to one or more variable values, and
applying the machine learning model to the one or more variable values to at least one of: identifying one or more microbial species that are associated with the one or more variable values; identifying one or more environmental characteristics that are associated with the one or more variable values; and/or identifying at least one microbial consortium that is associated with the one or more variable values.

10. The method of claim 9, wherein the one or more variable values may include at least one of a phenotype of a microbe, a desired amount of nitrogen fixation, a desired amount of carbon sequestration, one or more environmental sample characteristics, or one or more variables.

11. The method of claim 9, wherein the one or more environmental characteristics include an environmental source location and an environmental composition.

12. The method of claim 8, wherein the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

13. The method of claim 1, wherein the one or more environmental conditions include at least one of a particular concentration of N₂ gas, a particular concentration of CO₂ gas, availability of one or more specific nutrients, availability of one or more specific salts, or availability of one or more specific additives.

14. The method of claim 1, wherein the one or more variable measurements includes a variable measurement that indicates an increase in carbon sequestration, an increase in nitrogen fixation, an increase in biomass, or having a microbe that is able to meet a particular survival time.

15. The method of claim 1, wherein a successful microbial biomining result is produced when each variable measurement of one or more variable measurements at least met a corresponding variable measurement threshold.

16. The method of claim 1, wherein the identification information of a microbe includes a DNA biomarker of the microbe.

17. One or more non-transitory computer-readable media storing computer-executable instructions that upon execution cause one or more processors to perform acts comprising:
generating a machine learning model that based on training data that includes the identification information of one or more microbes, the machine learning model at least correlating one or more environmental sample variable values of multiple environmental samples with microbial variable values of one or more microbial species and one or more microbial consortia that are present in the multiple environmental samples;
receiving a request for information related to one or more variable values; and
applying the machine learning model to the one or more variable values to at least one of identifying one or more microbial species that are associated with the one or more variable values, identifying one or more environmental characteristics that are associated with the one or more variable values, or identifying at least one microbial consortium that is associated with the one or more variable values.

18. The one or more non-transitory computer-readable media of claim 17, wherein the one or more variable values may include at least one of a phenotype of a microbe, a desired amount of nitrogen fixation, a desired amount of carbon sequestration, one or more environmental sample characteristics, or one or more variables.

19. The one or more non-transitory computer-readable media of claim 17, wherein the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

20. A computing device, comprising:
one or more processors; and
memory including a plurality of computer-executable components that are executable by the one or more processors to perform a plurality of actions, the plurality of actions comprising:
generating a machine learning model that, based on training data that includes the identification information of one or more microbes, the machine learning model at least correlates one or more environmental sample variable values of multiple environmental samples with microbial variable values of one or more microbial species and one or more microbial consortia that are present in the multiple environmental samples;
receiving a request for information related to one or more variable values; and
applying the machine learning model to the one or more variable values to at least one of identifying one or more microbial species that are associated with the one or more variable values, identifying one or more environmental characteristics that are associated with the one or more variable values, or identifying at least one microbial consortium that is associated with the one or more variable values.

21. A method, comprising:
obtaining an environmental sample comprising organic matter for microbial biomining;
homogenizing the environmental sample to produce an input sample;
culturing the input sample in an environment that includes one or more environmental conditions; and
in response to determining based on one or more variable measurements that resulted from the culturing that the input sample produced a successful microbial biomining result, obtaining identification information for microbes that are present in a corresponding microbial consortium of the input sample.

22. The method of claim 21, further comprising in response to determining based on the one or more variable measurements that resulted from the culturing that the input sample produced an unsuccessful microbial biomining result, producing a second input sample based on the one or more selection criteria for testing.

23. The method of claim 21, further comprising:
producing a second input sample based on the one or more selection criteria for testing;
culturing the second input sample as selected in an environment that includes one or more environmental conditions; and
in response to determining based on one or more variable measurements that resulted from the culturing that the second input sample produced an additional successful microbial biomining result, obtaining additional identification information for additional microbes that are present in a second corresponding microbial consortium of the second input sample.

24. The method of claim 21, further comprising:
culturing the corresponding microbial consortium of the input sample into a microbial culture;
growing a selected culture portion of the microbial culture in the environment that includes one or more environmental conditions; and
in response to determining based on one or more variable measurements of the selected culture portion that the selected culture portion produced a successful microbial biomining result, obtaining additional identification information for additional microbes that are present in an additional corresponding microbial consortium of the selected culture portion.

25. The method of claim 24, further comprising in response to determining based on one or more variable measurements of the selected culture portion that the culture portion produced an unsuccessful microbial biomining result, selecting an additional culture portion of the microbial culture for testing.

26. The method of claim 24, further comprising:
growing an additional selected culture portion of the microbial culture in the environment that includes one or more environmental conditions; and
in response to determining based on one or more variable measurements of the additional selected culture portion that the selected culture portion produced a successful microbial biomining result, obtaining further identification information for further microbes that are present in a further corresponding microbial consortium of the additional selected culture portion.

27. The method of claim 24, further comprising generating a machine learning model based on training data that includes the identification information and the additional identification information.

28. The method of claim 27, wherein the machine learning model at least correlates one or more environmental sample variable values of the environmental sample with microbial variable values of one or more microbial species and one or more microbial consortia that are present in the environmental sample.

29. The method of claim 28, further comprising:
receiving a request for information related to one or more variable values, and
applying the machine learning model to the one or more variable values to at least one of; identifying one or more microbial species that are associated with the one or more variable values; identifying one or more environmental characteristics that are associated with the one or more variable values; and/or identifying at least one microbial consortium that is associated with the one or more variable values.

30. The method of claim 29, wherein the one or more variable values may include at least one of a phenotype of a microbe, a desired amount of nitrogen fixation, a desired amount of carbon sequestration, one or more environmental sample characteristics, and/or one or more variables.

31. The method of claim 29, wherein the one or more environmental characteristics include an environmental source location and an environmental composition.

32. The method of claim 28, wherein the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

33. The method of claim 21, wherein the one or more environmental conditions include at least one of a particular concentration of N₂ gas, a particular concentration of CO₂ gas, availability of one or more specific nutrients, availability of one or more specific salts, or availability of one or more specific additives.

34. The method of claim 21, wherein the one or more variable measurements includes a variable measurement that indicates an increase in carbon sequestration, an increase in nitrogen fixation, an increase in biomass, or having a microbe that is able to meet a particular survival time.

35. The method of claim 21, wherein a successful microbial biomining result is produced when each variable measurement of one or more variable measurements at least met a corresponding variable measurement threshold.

36. The method of claim 21, wherein the identification information of a microbe includes a DNA biomarker of the microbe.

37. One or more non-transitory computer-readable media storing computer-executable instructions that upon execution cause one or more processors to perform acts comprising:
generating a machine learning model that based on training data that includes the identification information of one or more microbes, the machine learning model at least correlating one or more environmental sample variable values of an environmental sample with microbial variable values of one or more microbial species and one or more microbial consortia that are present in the environmental sample;
receiving a request for information related to one or more variable values; and
applying the machine learning model to the one or more variable values to at least one of: identifying one or more microbial species that are associated with the one or more variable values; identifying one or more environmental characteristics that are associated with the one or more variable values; and/or identifying at least one microbial consortium that is associated with the one or more variable values.

38. The one or more non-transitory computer-readable media of claim 37, wherein the one or more variable values may include at least one of a phenotype of a microbe, a desired amount of nitrogen fixation, a desired amount of carbon sequestration, one or more environmental sample characteristics, or one or more variables.

39. The one or more non-transitory computer-readable media of claim 37, wherein the machine learning model further correlates one or more environmental sample variable values and microbial variable values with one or more variable values, and wherein the one or more variable values include at least an absolute amount of CO₂ sequestered by a biomass, a ratio of biomass to sequestered CO₂, an amount of time that CO₂ is sequestered by the biomass, an absolute amount of nitrogen fixation by the biomass, a mass ratio of the biomass to an absolute amount fixed nitrogen, a total profit derived from CO₂ sequestration, a ratio of food mass produced to mass of CO₂ sequestered by the biomass, or an amount of time that CO₂ is sequestered by the biomass.

40. A computing device, comprising:
one or more processors; and
a memory including a plurality of computer-executable components that are executable by the one or more processors to perform a plurality of actions, the plurality of actions comprising:
generating a machine learning model that, based on training data that includes the identification information of one or more microbes, the machine learning model at least correlates one or more environmental sample variable values of an environmental sample with microbial variable values of one or more microbial species and one or more microbial consortia that are present in the environmental sample;
receiving a request for information related to one or more variable values; and
applying the machine learning model to the one or more variable values to at least one of: identifying one or more microbial species that are associated with the one or more variable values; identifying one or more environmental characteristics that are associated with the one or more variable values; and/or identifying at least one microbial consortium that is associated with the one or more variable values.

41. A method for producing a microbial consortium that performs a specified function, the method comprising:
providing a sample comprising a plurality of microorganisms;
inoculating a first volume of a growth medium with a portion of said sample to provide a first culture;
growing the first culture under a set of selective conditions;
producing a first taxonomic classification of microorganisms in the first culture;
inoculating a second volume of the growth medium with a portion of the first culture to provide a second culture;
growing the second culture under the set of selective conditions;
producing a second taxonomic classification of microorganisms in the second culture;
deriving a measure of microbial community stability of the second culture with respect to the first culture using the second taxonomic classification and the first taxonomic classification.

42. A method for producing a microbial consortium that performs a specified function, the method comprising:
a) providing a sample comprising a plurality of microorganisms;
b) inoculating an Nth volume of a growth medium with a portion of said sample to provide an Nth culture;
c) growing the Nth culture under a set of selective conditions;
d) producing an Nth taxonomic classification of microorganisms in the Nth culture;
e) inoculating a N+1th volume of the growth medium with a portion of the Nth culture;
growing the N+1th culture under the set of selective conditions;
g) producing a N+1th taxonomic classification of microorganisms in the N+1th culture;
h) deriving a measure of microbial community stability of the N+1th culture with respect to the Nth culture using the N+1th taxonomic classification and the Nth taxonomic classification;
i) repeating iteratively steps (e) to (h) with the N+1th culture acting as the Nth culture until the measure of microbial community stability reaches a plateau value; and
j) providing the stable N+1th culture as comprising a microbial consortium that performs a specified function.

43. The method of claim 42, wherein the sample is an environmental sample.

44. The method of claim 43, wherein the environmental sample is a soil or water sample.

45. The method of claim 42, wherein the growth medium and/or selective conditions select for the specified function.

46. The method of claim 42, wherein producing a taxonomic classification comprises obtaining metagenomic nucleotide sequence data for a culture and identifying taxonomic units present in the culture using analysis of the metagenomic nucleotide sequence data.

47. The method of claim 42, wherein the microbial consortium comprises a number of taxonomic units that is at least 2, 3, 4, 5, or 6.

48. The method of claim 47, wherein a microbial community having a number of taxonomic units that is less than the number of taxonomic units of the microbial consortium does not perform the specified function.

49. The method of claim 47, wherein any one of the taxonomic units alone does not perform the specified function.

50. The method of claim 42, wherein the measure of microbial community stability comprises a measure of richness, diversity, abundance, and/or membership.

51. The method of claim 42, wherein the growing occurs for an empirically determined time for growth to end of exponential phase.

52. The method of claim 42, further comprising measuring the growth rate of the Nth or N+1th culture.

53. The method of claim 52, wherein a growth rate is determined by measuring cell mass as a function of time.

54. The method of claim 57, wherein at least one of the taxonomic units does not grow as a pure culture in the culture medium under the selective conditions.

55. The method of claim 57, wherein a microbial community comprising a number of taxonomic units that is at least two and that is less than the number of taxonomic units of the microbial consortium does not grow in the culture medium under the selective conditions.
